(19) United States
(12) Patent Application Publication
(10) Pub. No.: US 2020/0407752 A1
(43) Pub. Date: Dec. 31, 2020

(54) MICROVESICLE AND METHOD FOR PRODUCING THE SAME

(71) Applicants: Zhong LI, (US); Misako KATSURA, (US)

(72) Inventors: Zhong LI, Chuo-ku, Tokyo (JP); Misako KATSURA, Chuo-ku, Tokyo (JP); Luo FENG, San Gabriel, CA (US)

(73) Assignees: Zhong LI, Chuo-ku, Tokyo (JP); Misako KATSURA, Chuo-ku, Tokyo (JP)

(21) Appl. No.: 17/001,319

(22) Filed: Aug. 24, 2020

Related U.S. Application Data
(63) Continuation of application No. 14/775,458, filed on Sep. 11, 2015, now abandoned.
(60) Provisional application No. 61/894,563, filed on Oct. 23, 2013.
(60) Provisional application No. 61/779,556, filed on Mar. 13, 2013.
(60) Provisional application No. 61/894,563, filed on Oct. 23, 2013.

(51) Int. Cl. C12N 15/86; A61K 48/00

(52) U.S. Cl. CPC C12N 15/86; A61K 48/0058; C12N 2740/15071; C12N 2830/008; C12N 2740/15043

(57) ABSTRACT

The present invention provides a method for producing microvesicles comprising a transgene product and/or a lentiviral RNA comprising a transgene, comprising the steps of: culturing a cell into which the transgene has been introduced using a lentiviral vector in vitro to extracellularly release microvesicles comprising the transgene product and/or the lentiviral RNA comprising the transgene, wherein said lentiviral vector is deficient in at least one structural protein gene and comprises the transgene under control of a telomerase reverse transcriptase (TERT) gene promoter in a lentiviral genome sequence, and collecting the microvesicles released; and a microvesicle obtained according to this method and its use.

### TECHNICAL FIELD

The present invention relates to a microvesicle and a method for producing the same.

### BACKGROUND ART

Various cells are known to secrete or release microvesicles (small membrane vesicles), for example, exosomes in vivo. It has been thought that one of the roles of the microvesicles is to extracellularly release unnecessary intracellular components. In recent years, however, the possibility has been indicated that the microvesicles serve as signaling vehicles for transmitting substances such as proteins or lipids between secreting cells and their target cells and function in cell-cell interaction.

Diverse clinical applications of microvesicles, particularly, exosomes, have been proposed so far. For example, Patent Literature 1 discloses use of an exosome isolated from reticulocytes comprising a Plasmodium sp. antigen in defense against malaria. Patent Literature 2 discloses the treatment of cancer using an exosome from a B cell. Patent Literature 3 discloses use of a stem-cell derived microvesicle in endothelial or epithelial regeneration.

Lentivirus, for example, human immunodeficiency virus (HIV) infects cells and has the property of being integrated into the genomes of both dividing and non-dividing cells. Therefore, lentiviral vectors based on a lentiviral genome sequence are widely used as a tool for gene transduction.

### CITATION LIST

### Patent Literature

Patent Literature 1: International Publication WO2011/080271

Patent Literature 2: International Publication WO2011/000551

Patent Literature 3: International Publication WO2009/057165

### SUMMARY OF INVENTION

### Technical Problem

An object of the present invention is to provide a microvesicle and a method for producing the same.

### Solution to Problem

The present inventors have conducted diligent studies to attain the object and consequently found that the introduction of a transgene into cells using a lentiviral vector comprising the transgene under control of a telomerase reverse transcriptase (TERT) gene promoter can activate the integration of the transgene into host genome and its expression and can enhance the extracellular release of microvesicles having transgene products, etc., produced by the cells. On the basis of these findings, the present invention has been completed.

Specifically, the present invention encompasses the followings:

[1] A method for producing microvesicles comprising a transgene product and/or a lentiviral RNA comprising a transgene, comprising the steps of: culturing a cell into which the transgene has been introduced using a lentiviral vector in vitro to extracellularly release microvesicles comprising the transgene product and/or the lentiviral RNA comprising the transgene, wherein said lentiviral vector is deficient in at least one structural protein gene and comprises the transgene under control of a telomerase reverse transcriptase (TERT) gene promoter in a lentiviral genome sequence, and collecting the microvesicles released.

[2] The method according to [1], wherein said cell does not have said at least one structural protein gene.

[3] The method according to [1] or [2], wherein said lentiviral vector is deficient in env gene.

[4] The method according to any of [1] to [3], wherein said telomerase reverse transcriptase (TERT) gene promoter is a human TERT gene promoter.

[5] The method according to [4], wherein said human TERT gene promoter comprises the nucleotide sequence of SEQ ID NO: 1 or a nucleotide sequence having 90% or more sequence identity to the nucleotide sequence of SEQ ID NO: 1.

[6] The method according to [5], wherein said human TERT gene promoter comprises a nucleotide sequence having 95% or more sequence identity to the nucleotide sequence of SEQ ID NO: 1.

[7] The method according to any of [1] to [6], wherein said lentiviral RNA comprises a TERT gene promoter sequence upstream of the transgene.

[8] The method according to any of [1] to [7], wherein said lentiviral vector is:
(i) an RNA vector comprising the lentiviral genome sequence,
(ii) a DNA vector encoding an RNA comprising the lentiviral genome sequence, or
(iii) a viral particle carrying an RNA comprising the lentiviral genome sequence.

[9] The method according to any of [1] to [8], wherein said lentiviral genome sequence comprises at least a portion of TERT transcribed region between the TERT gene promoter and the transgene.

[10] The method according to [9], wherein said at least a portion of TERT transcribed region comprises the nucleotide sequence of SEQ ID NO: 2 or a nucleotide sequence having 90% or more sequence identity to the nucleotide sequence of SEQ ID NO: 2.

[11] The method according to any of [1] to [10], wherein said lentiviral genome sequence is an HIV genome sequence.

[12] The method according to [11], wherein said HIV genome sequence is an HIV-1 genome sequence.

[13] The method according to [12], wherein said HIV-1 genome sequence comprises 5′ LTR; packaging signal w; gag gene; pol gene; vif gene; vpr gene; tat gene; rev gene; vpu gene; and 3′ LTR.

[14] The method according to any of [1] to [13], wherein said transgene encodes a protein or RNA.

[15] The method according to any of [1] to [14], wherein said lentiviral vector comprises said transgene being a tumor-suppressor gene.

[16] The method according to [15], wherein said tumor-suppressor gene is PTEN or p16 gene.

[17] The method according to any of [1] to [14], wherein said lentiviral vector comprises said transgene that encodes a shRNA.

[18] The method according to [17], wherein said shRNA targets a gene encoding a cell proliferation regulator.

[19] The method according to [18], wherein said cell proliferation regulator is CDC6.

[20] The method according to any of [1] to [19], wherein said cell is a human cell.

[21] The method according to any of [1] to [20], wherein said cell is a kidney-derived cell.

[22] The method according to any of [1] to [21], wherein said cell is human embryonic kidney 293T cell.

[23] A microvesicle comprising a transgene product and/or a lentiviral RNA comprising a transgene, wherein said microvesicle is produced by the method according to any of [1] to [22].

[24] A method of gene transduction comprising, contacting a target cell with the microvesicle comprising the transgene product and/or the lentiviral RNA comprising the transgene according to [23] to fuse them, thereby introducing the transgene into the cell.

[25] The method according to [24], wherein said target cell is contacted with the microvesicle in vitro.

[26] A composition comprising the microvesicle according to [23].

[27] A pharmaceutical composition comprising the microvesicle according to [23].

[28] The pharmaceutical composition according to [27], which is for use in treatment of cancer.

[29] The pharmaceutical composition according to [28], wherein said cancer is selected from the group consisting of colon cancer, pancreatic cancer, kidney cancer, lung cancer, neuroblastoma, breast cancer, ovarian cancer, gastric cancer, prostate cancer, thyroid cancer and malignant lymphoma.

[30] The pharmaceutical composition according to [28] or [29], wherein said cancer involves an elevated expression of CDC6.

[31] The pharmaceutical composition according to any of [27] to [30], further comprising a pharmaceutically acceptable carrier.

[32] A method for treating a patient, comprising administering the microvesicle according to [23] to said patient in need of introduction of said transgene or said transgene product.

[33] The method according to [32], wherein said patient suffers from cancer.

[34] The method according to [33], wherein said cancer is selected from the group consisting of colon cancer, pancreatic cancer, kidney cancer, lung cancer, neuroblastoma, breast cancer, ovarian cancer, gastric cancer, prostate cancer, thyroid cancer and malignant lymphoma.

[35] The method according to [33] or [34], wherein said cancer involves an elevated expression of CDC6.

This description includes the disclosures in U.S. Provisional Application Nos. 61/779,556 and U.S. 61/894,563, to which the present application claims priority.

### Effects of Invention

The present invention provides a microvesicle and a method for producing the same.

### BRIEF DESCRIPTION OF THE DRAWINGS

FIG. 1 is a schematic diagram of the HIV-1 genomic region of plasmids having an HIV-1 backbone. FIG. 1A is a schematic diagram of the HIV-1 genomic region of pNL4-3. FIG. 1B is a schematic diagram of the HIV-1 genomic region of pTHTK.

FIG. 2 shows recombinant lentiviral plasmid vectors pRBL0213T and pTHTN. FIG. 2A is an agarose gel electrophoretogram showing the plasmid DNA mapping of pRBL0213T and pTHTN. Lane 1: pRBL0213T, EcoR I; Lane 2: pRBL0213T, Mlu I+Xho I; Lane 3: pRBL0213T, NheI; Lane 4: pTHTN, EcoR I; Lane 5: pTHTN, Mlu I+Xho I; Lane M: 1 kb ladder. FIG. 2B is a schematic diagram of the HIV-1 genomic region of pRBL0213T. FIG. 2C is a schematic diagram of the HIV-1 genomic region of pTHTN.

FIG. 3 shows recombinant lentiviral plasmid vector pRBL001. FIG. 3A is an agarose gel electrophoretogram showing the plasmid DNA mapping of pRBL001. Lane 1: pRBL001, EcoR I; Lane 2: pRBL001, EcoR I+Nhe I; Lane 3: pRBL001, Mlu I+Xho I; Lane 4: pRBL001, Sal I; Lane 5: pRBL0213T, Nhe I; Lane M: 1 kb ladder. FIG. 3B is a schematic diagram of the HIV-1 genomic region of pRBL001. The restriction enzyme sites represent RI: EcoR I, Sal: Sal I, Mlu: Mlu I, Nhe: Nhe I and Xho: Xho I.

FIG. 4 is a schematic diagram of the HIV-1 genomic region of recombinant lentiviral plasmid vectors. FIG. 4A is a schematic diagram of the HIV-1 genomic region of pNL4-3 (wild-type HIV-1). FIG. 4B is a schematic diagram of the HIV-1 genomic region of pD64V that has a mutation in HIV-1 integrase and is deficient in the integration of HIV-1 virus genomic DNA into host genome. FIG. 4C is a schematic diagram of the HIV-1 genomic region of pTHTK. FIG. 4D is a schematic diagram of the HIV-1 genomic region of pTHTN. FIG. 4E is a schematic diagram of the HIV-1 genomic region of pTHTH that lacks the 5′ portion of hTERT promoter. FIG. 4F is a schematic diagram of the HIV-1 genomic region of pTHTC having a promoter sequence from human cytomegalovirus (CMV).

FIG. 5 is a schematic diagram showing the preparation of Bpm I site for detecting LTR-Tag. FIG. 5A is a schematic diagram showing the novel Bpm I site prepared in 5′ LTR of the HIV-1 genomic region of pTHTK. FIG. 5B is a schematic diagram showing that Bpm I restriction enzyme cleaves host chromosomal DNA at an integration site (14 nucleotides) adjacent to the Bpm I site copied to HIV-1 3′ LTR. FIG. 5C is a schematic diagram of the terminal structure of lentiviral genome.

FIG. 6 is a schematic diagram showing the formation of LTR-Tag via ligation-mediated PCR (LM-PCR).

FIG. 7 is a gel electrophoretogram of LM-PCR for detecting LTR-Tag.

FIG. 8 shows results of examining the splicing of viral RNA.

FIGS. 8A and 8B are photographs showing results of electrophoresing RT-PCR reaction products using 2.2% agarose gel in 1×TAE. FIG. 8A is a gel electrophoretogram showing PCR products obtained using primers 5′ LTRU5 and 3′ NL8960. FIG. 8B is a gel electrophoretogram showing PCR products obtained using primers 5′ LTRU5 and 3′ NL5850. The positions of bands of spliced RNA fragments a (E1+A1/3′NL5850), b (E1+A2/3′NL5850), c1 (E1+E2+E3+A3/3′NL5850), c2 (E1+E2+A3/3′NL5850) and c3 (E1+E3+A3/3′NL5850) are shown on the right side of FIG. 8B. FIG. 8C is a schematic diagram showing an unspliced transcript. FIG. 8D is a schematic diagram showing spliced RNA. In FIG. 8D, A1 to A3 denote 3′ splice acceptors and E1 to E3 denote exons.

FIG. 9 is a graph showing results of p24 assay.

FIG. 10 is a schematic diagram showing the constitution of plasmid vectors used in PTEN assay. FIG. 10A shows pGL3-1375 (Empt.; plasmid size: approximately 7 kb) as a negative control in PTEN gene transduction. FIG. 10B shows pcDNA3.1/CMV-hPTEN (Regul.; plasmid size: approximately 7.5 kb) as a positive control in PTEN gene transduction. FIG. 10C shows retroviral vector pRBL016Bn (Retro.; plasmid size: approximately 7.9 kb) for PTEN gene transduction. FIG. 10D shows recombinant lentiviral vector pRBL0213T (Lenti.; plasmid size: approximately 15 kb) for PTEN gene transduction. The restriction enzyme sites in the vectors represent BamH: BamH I; Bgl: Bgl II; RI: EcoR I; RV: EcoR V; Hd3: Hind III; Mlu: Mlu I; Nco: Nco I; Nde: Nde I; Sal: Sal I; Stu: Stu I; and Xho: Xho I.

FIG. 11 is a graph showing results of PTEN assay on transient transfection with a plasmid vector.

FIG. 12 is a graph showing the ratio of PTEN activity in the mv lysates to PTEN activity in the cell lysates shown in FIG. 11.

FIG. 13 is a graph showing results of PTEN assay on recombinant lentiviral particle RBL0213T infection.

FIG. 14 is a graph showing the ratio of PTEN activity in mv lysates to PTEN activity in cell lysates of cells transfected with recombinant lentiviral plasmid vector pRBL0213T or cells infected by recombinant lentiviral particle RBL0213T.

FIG. 15 is a photograph showing results of Western blot on viral proteins or endogenous or foreign proteins in mv or in cells incubated with my. FIG. 15A shows results obtained using anti-Vpu antibody. FIG. 15B shows results obtained using anti-RT antibody. FIG. 15C shows results obtained using anti-FEN-1 antibody.

FIG. 16 is a set of microscope photographs showing the delivery of contents to cells by mv in which c-myc-FEN-1 was encapsulated. FIG. 16A shows an anti-c-myc antibody stained image. FIG. 16B shows a DAPI stained image.

FIG. 16C shows an overlaid image of FIGS. 16A and 16B. FIG. 16D shows an image prepared from the overlaid image by the color curve program in the image “adjustment” method of Photoshop® (Adobe Systems Inc.).

FIG. 17 is a graph showing the cancer cell proliferation suppressive effect of Lenti-mv2010/CDC6 shRNA.

FIG. 18 shows a photograph indicating results of Western blotting analysis in cellular protein extracts and mv lysates.

FIGS. 19A-D show photographs showing typical morphologies observed for tumors from respective test groups by pathological examination. FIG. 19A, negative control group; FIG. 19B, interferon (IFN) α-2b injection group; FIG. 19C, Cytomox PTEN injection group; and FIG. 19D, Cytomox p53 injection group.

FIGS. 20A-D show photographs showing typical morphologies observed for tumors from respective test groups by pathological test. FIG. 20A, low dose Cytomox EX injection group; FIG. 20B, high dose Cytomox EX injection group;

FIG. 20C, low dose Cytomox HD injection group; and FIG. 20D, low dose Cytomox HD injection group.

### DETAILED DESCRIPTION OF THE PREFERRED EMBODIMENTS

Hereinafter, the present invention will be described in detail.

### 1. Method for Producing Microvesicles

The present invention relates to a method for producing microvesicles. The method of the present invention comprises the steps of: culturing a cell into which the transgene has been introduced using a lentiviral vector in vitro to extracellularly release microvesicles comprising the transgene product and/or the lentiviral RNA comprising the transgene, wherein said lentiviral vector is deficient in at least one structural protein gene and comprises the transgene under control of a telomerase reverse transcriptase (TERT) gene promoter in a lentiviral genome sequence, and collecting the microvesicles released.

The microvesicles produced by the method of the present invention comprise a transgene product and/or a lentiviral RNA comprising a transgene. The method of the present invention can efficiently produce microvesicles comprising a transgene product and/or a lentiviral RNA comprising a transgene.

The lentiviral vector according to the present invention refers to a vector for gene transduction having a lentiviral genome sequence as a basic backbone. Lentivirus is an RNA virus having reverse transcriptase. The lentivirus can integrate viral genomic DNA (proviral DNA) into the host chromosomes of not only dividing cells but non-dividing cells so that virus-derived genes can be expressed by the host cells. The lentiviral vector is based on such properties of the lentivirus.

The lentiviral vector according to the present invention can be:
(i) an RNA vector comprising the lentiviral genome sequence,
(ii) a DNA vector encoding an RNA comprising the lentiviral genome sequence, or
(iii) a viral particle carrying an RNA comprising the lentiviral genome sequence.

The RNA vector of (i) can be prepared, for example, by in vitro transcription from an expression vector or an expression cassette comprising the lentiviral genome sequence. The DNA vector of (ii) can include a plasmid vector. The plasmid vector usually comprises a DNA sequence encoding the RNA comprising the lentiviral genome sequence as well as a promoter and a terminator for bringing about the transcription of the DNA sequence, a replication origin, and a marker gene for screening for recombinants, etc. Such a DNA vector can be prepared by a method known in the art using a gene recombination technique or the like. The viral particle of (iii) may be a viral particle pseudotyped by an envelope protein of a different virus, for example, envelope glycoprotein G (VSV-G) of vesicular stomatitis virus (VSV). The pseudotyped viral particle may be prepared, for example, by: cotransfecting a cultured cell with a DNA vector encoding an RNA comprising the lentiviral genome sequence and a plasmid encoding the envelope protein (e.g., VSV-G) of the different virus; collecting a viral particle released into a medium; and purifying the particle.

The lentiviral vector used in the present invention is deficient in at least one viral structural protein gene in its lentiviral genome sequence. Such a lentiviral genome sequence may be one in which at least one viral structural protein gene has been disrupted (e.g., by the deletion of a partial or whole region or by the insertion of a nucleic acid molecule) in a full-length lentiviral genome sequence. In the present invention, the term “deficient” in a gene means that the whole gene is deleted or the gene is disrupted or mutated so that a functional protein cannot be expressed. The viral structural protein gene in which the lentiviral vector is deficient may be at least one selected from the group consisting of gag, pol and env genes. The gag gene encodes a protein involved in viral particle formation. The pol gene encodes an enzyme such as reverse transcriptase (RT). The env gene encodes a coat (envelope) protein involved in adsorption on and penetration to host cells. For example, the lentiviral vector may be deficient in env gene. For example, a region corresponding to position 6344 to position 7611 of SEQ ID NO: 4 may be deleted from the lentiviral vector to result in the deficiency of HIV-1 env gene. In one embodiment, the cell does not have, in the genome or outside the chromosome, the at least one viral structural protein gene in which the lentiviral vector is deficient. Such a cell into which the transgene has been introduced using the lentiviral vector does not produce infectious lentiviral particles. Therefore, produced microvesicles are highly safe.

Examples of the lentiviral genome sequence used as a basic backbone in the lentiviral vector according to the present invention include, but not limited to, sequences from the genomes of human immunodeficiency virus (HIV), simian immunodeficiency virus (SIV), feline immunodeficiency virus (FIV) and canine immunodeficiency virus (CIV). The lentiviral genome sequence according to the present invention is preferably an HIV genome sequence. More specifically, the HIV genome sequence may be HIV-1 or HIV-2 genome sequence. Preferably, the HIV genome sequence may be HIV-1 genome sequence. The HIV may be a strain belonging to HIV-1 group M, N, O or P. More specifically, the HIV may be any of HIV strains including HIV-1 IIIb, HIV-1 SF2, HIV-1 SF162, HIV-1 BRU, HIV-1 NY5, HIV-1L AI, HIV-1 NL4-3, etc. An exemplary HIV genome sequence is available from GenBank accession Nos. EU541617, K03455 and K02013, etc. The lentiviral genome sequence may be RNA or may be DNA.

The lentiviral genome sequence in the lentiviral vector may comprise 5′ LTR and 3′ LTR, and optionally at least one selected from the group consisting of packaging signal w, gag gene, pol gene, vif gene, vpr gene, tat gene, rev gene, vpu gene or vpx gene, nef gene and env gene. In a preferred embodiment, the lentiviral genome sequence of HIV-1 in the lentiviral vector may comprise 5′ LTR, packaging signal w, gag gene, pol gene, vif gene, vpr gene, tat gene, rev gene, vpu gene and 3′ LTR. In another preferred embodiment, the lentiviral genome sequence of HIV-2 in the lentiviral vector may comprise 5′ LTR, packaging signal w, gag gene, pol gene, vif gene, vpr gene, tat gene, rev gene, vpx gene and 3′ LTR. Such a lentiviral genome sequence typically comprises at least one splice donor (SD) and splice acceptor (SA). In one embodiment, the lentiviral genome sequence in the lentiviral vector may comprise vpu gene, tat gene and rev gene. The lentiviral genome sequence in the lentiviral vector may be deficient in nef gene.

The lentiviral vector according to the present invention comprises the transgene under control of a telomerase reverse transcriptase (TERT) gene promoter in a lentiviral genome sequence. TERT is an enzyme that synthesizes telomeric repeat during DNA replication. The telomerase reverse transcriptase (TERT) gene promoter used in the present invention may be, but not limited to, a human TERT gene promoter. Preferably, the human TERT gene promoter may comprise the nucleotide sequence of SEQ ID NO: 1 or a nucleotide sequence having 90% or more sequence identity to the nucleotide sequence of SEQ ID NO: 1. More preferably, the human TERT gene promoter may comprise a nucleotide sequence having 95%, 97%, 99%, 99.5% or 99.9% or more sequence identity to the nucleotide sequence of SEQ ID NO: 1.

In one embodiment, also preferably, the lentiviral vector according to the present invention comprises the nucleotide sequence of SEQ ID NO: 5 or a sequence having 90% or more, preferably 95% or more, more preferably 99% or more, for example, 99.8% or more sequence identity thereto as the lentiviral genome sequence; and a sequence comprising the TERT gene promoter and the transgene under control thereof, which has been further inserted into the lentiviral genome sequence (preferably in nef gene).

In the present invention, the phrase “transgene under control of a TERT gene promoter” means that the transcription of the transgene is initiated by the activity of the TERT gene promoter. Preferably, the transgene is located downstream of the TERT gene promoter.

At least a portion of TERT transcribed region may exist between the TERT gene promoter and the transgene inserted in the lentiviral genome sequence. The at least a portion of TERT transcribed region may comprise at least the first exon of TERT gene. The at least a portion of TERT transcribed region may comprise the nucleotide sequence of SEQ ID NO: 2 or a nucleotide sequence having 90%, 95%, 97%, 99% or 99.5% or more sequence identity to the nucleotide sequence of SEQ ID NO: 2.

The transgene used in the present invention may encode any protein or RNA such as microRNA (miRNA), small interfering RNA (siRNA) or short hairpin RNA (shRNA). In one embodiment, the transgene may be a tumor-suppressor gene. Examples of the tumor-suppressor gene include, but not limited to, tumor-suppressor genes known to those skilled in the art, such as p53, BRCA1, Rb, PTEN and p16 genes. Preferably, the tumor-suppressor gene may be PTEN or p16 gene. The PTEN protein is a phosphatase which catalyzes the dephosphorylation of phosphatidylinositol 3,4,5-triphosphate (PtdIns(3,4,5)P3). The PTEN gene may comprise the nucleotide sequence of SEQ ID NO: 6 or a nucleotide sequence having 90% or more, preferably 95% or more, more preferably 99% or more, for example, 99.5% or 99.9% or more sequence identity thereto. The PTEN gene also may be a nucleic acid encoding a PTEN protein that consists of the amino acid sequence of SEQ ID NO: 22 (GenBank accession Nos. AAD13528 and NP_000305) or an amino acid sequence having 90% or more, preferably 95% or more, more preferably 99% or more, for example, 99.5% or 99.7% or more sequence identity thereto. p16 protein (also referred to as p16^{INK4a}) is known as a cyclin-dependent kinase (CDK) inhibitor. For example, p16 gene may comprise the nucleotide sequence of SEQ ID NO: 23 (GenBank accession No. L27211) or a nucleotide sequence having 80% or more, preferably 90% or more, more preferably 95% or more, further preferably 99% or more, for example, 99.5% or 99.9% or more sequence identity thereto. p16 gene also may be a nucleic acid encoding a protein consisting of the amino acid sequence of SEQ ID NO: 24 or an amino acid sequence having 90% or more, preferably 95% or more, more preferably 99% or more sequence identity thereto. p16 gene preferably encodes a protein having CDK inhibitory activity. Examples of p16 gene include a nucleic acid comprising the nucleotide sequence of positions 434 to 480 of SEQ ID NO: 25. Further, p53 gene may comprise the nucleotide sequence of SEQ ID NO: 26 (GenBank accession No. BC003596) or an amino acid sequence having 80% or more, preferably 90% or more, more preferably 95% or more, further preferably 99% or more, for example, 99.5% or 99.9% or more sequence identity thereto. p53 gene also may be a nucleic acid encoding a protein consisting of the amino acid sequence of SEQ ID NO: 27 or an amino acid sequence having 90% or more, preferably 95% or more, more preferably 99% or more sequence identity thereto. p53 gene preferably encodes a protein having transcription factor activity. In the context of the present application, a nucleic acid may be DNA or RNA and may comprise a modified base.

In another embodiment, the transgene may encode a shRNA. The shRNA is a single-stranded RNA in which an antisense sequence complementary to a target sequence and a sense sequence (typically having a poly U overhang at the 3′ end) complementary to the antisense sequence are linked via a linker, and forms a hairpin structure via intramolecular double strand formation. Such a shRNA is intracellularly cleaved at its double-stranded portion into siRNAs, which can in turn cause RNAi to suppress the expression of a target gene comprising the target sequence. A shRNA precursor may be transcribed from the transgene and then subjected to editing and processing to form a shRNA. Even in that case, it is defined herein that such a transgene encodes a shRNA. A target of the shRNA may include, but not limited to, a gene encoding a cell proliferation regulator. Examples of the cell proliferation regulator include proteins involved in DNA replication or the regulation of cell cycle, such as CDC6, cyclin E, CDK2, CDT1, ORC2 and MCM7. Preferably, the cell proliferation regulator may be CDC6. The CDC6 is a protein that plays a central role in the initiation of DNA replication. CDC6 knockdown has been found to result in the apoptosis of human cancer cells (Feng, et al., Cancer Res., 2003, Vol. 63, p. 7356-7364; Lau et al., EMBO Rep., 2006, Vol. 7, p. 425-430; and Feng et al., Mol. Cell. Biochem., 2008, Vol. 311, p. 189-197). CDC6 gene may comprise the nucleotide sequence of SEQ ID NO: 7 or a nucleotide sequence having 90% or more, preferably 95% or more, more preferably 99% or more, for example 99.5% or 99.9% or more sequence identity thereto. The transgene encoding CDC6 shRNA may comprise a DNA sequence consisting of the nucleotide sequence of SEQ ID NO: 10 or a nucleotide sequence having 90% or more, preferably 95% or more, more preferably 98% or more sequence identity thereto. Typically, the CDC6 shRNA comprises: an antisense sequence of CDC6 shRNA consisting of the nucleotide sequence of SEQ ID NO: 19 or a nucleotide sequence having 90% or more sequence identity thereto; a linker consisting of the nucleotide sequence of SEQ ID NO: 20 or a nucleotide sequence having 80% or more sequence identity thereto; and a sense sequence consisting of a sequence complementary to the antisense sequence and a 3′ poly U overhang of two or more bases. The transgene product may comprise an RNA from the transgene as described above or a protein translated from the RNA from the transgene.

Each lentiviral vector may comprise one or more transgenes. Two or more transgenes may be introduced into a cell using one lentiviral vector or using two or more lentiviral vectors. For example, the lentivirus vector may contain a transgene that encodes shRNA and a transgene that is a tumor-suppressor gene. In a preferred embodiment, the lentiviral vector comprises a transgene encoding CDC6 shRNA and/or a transgene encoding p16 protein.

The transgene according to the present invention is introduced into a cell using the lentiviral vector in vitro. The organism species of the cell used in the present invention is preferably the same species as a recipient of the microvesicles to be produced. The cell may be, but not limited to, a mammalian cell, for example, a cell of dog, cat, cattle, sheep, mouse, rat or primate such as monkey or human. A human cell is preferred. Also preferably, the cell may be a kidney-derived cell, a uterus-derived cell, a lymphocyte-derived cell or a fibroblast cell. The cell may include human embryonic kidney 293T cells, human uterine cervix cancer HeLa cells, human lymphocyte CEM cells, N144 fibroblast cells and other human cell lines. Preferably, the cell may be human embryonic kidney 293T cell or human uterine cervix cancer HeLa cell.

The introduction of the transgene into the cell using the lentiviral vector can be performed by a method known in the art. The introduction of the transgene into the cell using the RNA vector or the DNA vector as the lentiviral vector may be performed by calcium phosphate method, lipofection method, DEAE dextran method or electroporation method or the like known in the art and may be performed using commercially available transfection reagents such as Lipofectamine® 2000 (Invitrogen) and FuGene® 6 (Roche). The introduction of the transgene into the cell using the viral particle as the lentiviral vector can be performed by adding the viral particle into a culture medium of the cell to infect the cell by the virus.

In addition to the lentiviral vector, an additional expression vector may also be introduced into the cell. Such an expression vector may comprise a transgene encoding a protein or RNA to be further encapsulated in the microvesicles, under control of a promoter. The cell into which the transgene has been thus introduced is then cultured. The culture may be performed by an appropriate method according to the cell. For example, the culture may be performed for 1 to 5 days, for example, 2 to 4 days, specifically 36 hours to 96 hours or 36 hours to 72 hours, in DMEM/high-glucose complete medium supplemented with 10% fetal bovine serum and an appropriate antibiotic. In the cell into which the transgene has been introduced using the lentiviral vector, lentiviral DNA (e.g., produced from the lentiviral RNA by the action of the reverse transcriptase) is integrated into intracellular genome by the action of integrase and the like to form proviral DNA. From this proviral DNA, the lentiviral RNA is produced by transcription mediated by intracellular RNA polymerase II. From the lentiviral RNA thus produced, the transgene product is usually produced through RNA splicing and/or protein translation, etc. The transgene product and/or the lentiviral RNA thus produced in the cell are incorporated into the microvesicles. The cell extracellularly releases such microvesicles during the culture so that the microvesicles accumulate in the medium. Preferably, the lentiviral vector used in the present invention is deficient in the structural protein gene, thereby forming no virus-like particle and allowing no virus-like particle to accumulate in the medium.

The method for producing microvesicles according to the present invention comprises the step of collecting the microvesicles released. The collection of the microvesicles can be performed by collecting the cell culture medium. The microvesicles thus collected may be further purified. For example, larger vesicles can be precipitated and removed by centrifugation at 1,000 to 10,000×g (in a preferred embodiment, 9,000×g), thereby purifying the microvesicles collected. Such centrifugation may be performed one or more times (preferably with different centrifugal forces). Alternatively or additionally, for example, the microvesicles can be purified by ultrafiltration using a membrane with a molecular weight cutoff of 1,000 kDa. Further, the microvesicles can also be precipitated by mixing with a PEG/NaCl solution and centrifugation, thereby purifying the microvesicles. After such purification, the microvesicles may be PEGylated. The PEGylation of the microvesicles can be performed using various types of PEGylating reagents and can be performed using, for example, methoxy PEG succinimidyl carbonate NHS (mPEG-NHS) (Croyle et al., J. Virol., 2004, Vol. 78, p. 912-921). For example, methoxy PEG succinimidyl carbonate NHS (mPEG-NHS, m.w. 10K (NANOCS, USA)) is added into the solution containing microvesicles and the mixture can be incubated at room temperature for 60 minutes on a rotary platform, thereby PEGylating the microvesicles. The microvesicles PEGylated or unPEGylated may then be subjected to buffer replacement by dialysis (e.g., using 1×PBS), concentration by ultrafiltration and filtration through a syringe filter (e.g., having 0.45-μm pore size), etc.

The microvesicles produced by the method of the present invention comprise the transgene product and/or the lentiviral RNA comprising the transgene. The lentiviral RNA may comprise a TERT gene promoter sequence upstream of the transgene. The transgene is introduced into a cell using the lentiviral vector comprising the TERT gene promoter sequence, thereby promoting the integration of the transgene into host genome and its expression and further enhancing the release of microvesicles from the cell. Therefore, the microvesicles can be efficiently produced.

In one embodiment, the present invention also relates to: a method for producing microvesicles comprising a transgene product and/or a lentiviral RNA comprising a transgene, comprising the steps of: introducing the transgene into a cell using a lentiviral vector in vitro, wherein said lentiviral vector is deficient in at least one structural protein gene and comprises the transgene under control of a telomerase reverse transcriptase (TERT) gene promoter in a lentiviral genome sequence,

culturing the cell to extracellularly release microvesicles comprising the transgene product and/or the lentiviral RNA comprising the transgene, and collecting the microvesicles released.

### 2. Microvesicle Produced by Method of the Present Invention

The present invention also relates to a microvesicle comprising a transgene product and/or a lentiviral RNA comprising a transgene, wherein the microvesicle is produced by the method of the present invention.

The microvesicle (my) according to the present invention refers to a membrane vesicle of 5 nm to 5 μm, preferably 10 nm to 1 μm, more preferably 20 nm to 500 nm in size that is produced by cells and extracellularly released or shed. The size of the microvesicle can be determined by an electron microscope method. Examples of the microvesicle generally include, but not limited to, exosomes, shedding microvesicles and apoptotic bodies. Typically, the microvesicle of the present invention is an exosome. The exosome is a membrane vesicle composed of a lipid bilayer. The exosome has a size of 150 nm or smaller, typically, 20 to 120 nm or 40 to 100 nm. The exosome is extracellularly secreted and produced by exocytosis resulting from the cell membrane fusion of multivesicular body (MVB) formed via the inward budding of an endosomal membrane.

The microvesicle according to the present invention may comprise a protein and/or RNA, such as the transgene product and/or the lentiviral RNA comprising the transgene, which has been transported from a host cell and encapsulated therein. Examples of the RNA that may be contained in the microvesicle include, but not limited to, mRNA, miRNA, shRNA, siRNA and lentiviral RNA (including various splicing variants of the lentiviral RNA). Examples of the protein that may be contained in the microvesicle include, but not limited to, viral proteins (e.g., HIV-1 Vpu protein and reverse transcriptase (RT) protein), cell-endogenous proteins (e.g., cytoskeletal proteins, signaling proteins and enzymes) and foreign transgene products. For example, the exosome may generally comprise a cytoskeletal protein (e.g., tubulin, actin and actin-binding protein), a membrane transport-related protein (e.g., annexin and Rab protein), a signaling protein (e.g., protein kinase and 14-3-3), a metabolic enzyme (e.g., GAPDH, ATPase and enolase), tetraspanin family (e.g., CD9, CD63, CD81 and CD82), a heat shock protein (e.g., HSP90 and HSP70), a MVB biosynthesis protein (e.g., Alix and TSG101), an immunomodulatory molecule (e.g., MHCI and MHCII), etc.

Lentiviral DNA (proviral DNA) comprising the lentiviral genome sequence comprising the transgene described above that has been introduced into a cell by the method of the present invention is integrated into host genome. The lentiviral RNA contained in the microvesicle of the present invention is transcribed from this proviral DNA. The lentiviral RNA may comprise an RNA sequence of the TERT gene promoter (e.g., hTERT promoter) at 5′ upstream of the transgene.

In a particularly preferred embodiment, the microvesicle of the present invention may comprise a tumor-suppressor protein such as PTEN and p16 protein and/or shRNA such as CDC6 shRNA or its precursor RNA, as the transgene product.

The microvesicle of the present invention can be taken up by other cells to deliver, into the cells, the transgene product and/or the lentiviral RNA comprising the transgene contained in the microvesicle.

### 3. Method of Gene Transduction Using Microvesicle of the Present Invention

The present invention also relates to a method of gene transduction comprising, contacting a target cell with the microvesicle comprising the transgene product and/or the lentiviral RNA comprising the transgene to fuse them, thereby introducing the transgene into the cell, wherein the microvesicle is produced by the method of the present invention. In one embodiment, the target cell can be contacted with the microvesicle in vitro or in vivo.

The microvesicle, particularly, the exosome, can penetrate into its neighboring other cells to participate in cell-cell interaction. The microvesicle is thought to be able to reach the inside of the target cell via membrane fusion or through an endocytosis-like manner, though the present invention is not restricted to this theory. In the method of gene transduction of the present invention, the contact of the target cell with the microvesicle may be made by any method known to those skilled in the art. For example, the in vitro contact of the target cell with the microvesicle may be made by the addition of the microvesicle into a cell culture medium. The in vivo contact of the target cell with the microvesicle may be made, for example, by the oral administration of the microvesicle or by the parenteral administration such as direct application or injection of the microvesicle to a target site (e.g., intrahepatic, intraarticular, intraventricular and intranasal sites). Other in vivo administration methods and administration sites that may be used will be described later in relation to administration methods and administration sites for a pharmaceutical composition.

The method of gene transduction of the present invention can efficiently deliver, into the target cell, the transgene product and/or the lentiviral RNA comprising the transgene contained in the microvesicle.

### 4. Composition Comprising Microvesicle of the Present Invention

The present invention also relates to a composition comprising the microvesicle produced by the method of the present invention. The composition can comprise any ingredient other than the microvesicle according to the intended use thereof. For example, the composition may be for use in gene transduction. In that case, the composition may comprise a drug promoting gene transduction and/or a drug stabilizing nucleic acid, etc.

### 5. Pharmaceutical Composition Comprising Microvesicle of the Present Invention and Treatment Method

The present invention also relates to a pharmaceutical composition comprising the microvesicle produced by the method of the present invention.

In one embodiment, the pharmaceutical composition may be for use in treatment of diseases such as cancer, diabetes, neurodegenerative disease, immune dysfunction, inflammation, liver cirrhosis, arteriosclerosis, thrombus and infection. Preferably, the pharmaceutical composition may be for use in treatment of cancer. More specifically, the cancer may be selected from the group consisting of, for example, colon cancer, pancreatic cancer, kidney cancer, lung cancer, neuroblastoma, breast cancer, ovarian cancer, gastric cancer, prostate cancer, thyroid cancer and malignant lymphoma. In one embodiment, the transgene product contained in the microvesicle may cause a reduced expression of CDC6. For example, the transgene product may be a shRNA targeting CDC6. In that case, the disease to be treated, for example, cancer, may involve an elevated expression of CDC6.

In the case of using the microvesicle in the pharmaceutical composition, the transgene or the transgene product contained in the microvesicle according to the present invention functions to prevent and/or treat the disease. For example, such a transgene may be a tumor-suppressor gene such as PTEN or p16 gene and/or may be a gene encoding a shRNA targeting a gene encoding a cell proliferation regulator or its precursor. In one embodiment, the transgene is PTEN gene and/or a gene encoding CDC6 shRNA. In this embodiment, the pharmaceutical composition may comprise, for example, the microvesicle produced by the method of the present invention in which the transgene is PTEN gene, and the microvesicle produced by the method of the present invention in which the transgene is a gene encoding CDC6 shRNA, in combination. In another embodiment, the transgene is p16 gene and/or a gene encoding CDC6 shRNA. In this embodiment, the pharmaceutical composition may comprise, for example, the microvesicle produced by the method of the present invention in which the transgene is p16 gene, and the microvesicle produced by the method of the present invention in which the transgene is a gene encoding CDC6 shRNA, in combination.

The pharmaceutical composition of the present invention comprises a liquid medium in addition to the microvesicle of the present invention. Examples of the liquid medium include water, physiologically acceptable buffer solutions (phosphate-buffered saline, etc.) and biocompatible aqueous mediums such as propylene glycol and polyoxyethylene sorbitan fatty acid ester. Such a medium is desirably sterilized and preferably adjusted to be isotonic to blood, if necessary.

The pharmaceutical composition may comprise a pharmaceutically acceptable carrier. The “pharmaceutically acceptable carrier” refers to an additive usually used in the field of pharmaceutical techniques. Examples of the pharmaceutically acceptable carrier include suspending agents, tonicity agents, buffers and preservatives. Such a carrier is used mainly for facilitating formulation and maintaining the dosage form and drug effects and may be appropriately used according to the need.

For example, glyceryl monostearate, aluminum monostearate, methylcellulose, carboxymethylcellulose, hydroxymethylcellulose and sodium lauryl sulfate can be used as the suspending agents. Examples of the tonicity agents include sodium chloride, glycerin and D-mannitol. Examples of the buffers include phosphate, acetate, carbonate and citrate buffer solutions. Examples of the preservatives include benzalkonium chloride, parahydroxybenzoic acid and chlorobutanol.

The pharmaceutical composition can also comprise, if necessary, a corrigent, a thickener, a solubilizing agent, a pH adjuster, a diluent, a surfactant, an expander, a stabilizer, an absorption promoter, a wetting agent, a humectant, an adsorbent, a coating agent, a colorant, an antioxidant, a flavoring agent, a sweetener, an excipient, a binder, a disintegrant, a disintegration inhibitor, a filler, an emulsifier, a flow control additive, a lubricant, or the like, in addition to those described above.

The pharmaceutical composition of the present invention can also contain an additional drug without losing pharmacological effects possessed by the microvesicle of the present invention. For example, the pharmaceutical composition may contain a predetermined amount of an antibiotic.

The dosage form of the pharmaceutical composition is not limited and can be any form that neither inactivates the microvesicle nor inactivates the transgene product and/or the lentiviral RNA contained in the microvesicle. The dosage form of the pharmaceutical composition may be, for example, a liquid, solid or semisolid form. Specific examples of the dosage form include: parenteral dosage forms such as injections, suspensions, emulsions, creams, eye drops, nasal drops, ointments, plasters, patches and suppositories; and oral dosage forms such as liquid formulations, capsules, sublingual formulations, troches, powders, tablets and granules. The dosage form of the pharmaceutical composition is preferably a liquid formulation such as an injection.

The pharmaceutical composition can be administered to an organism in a pharmaceutically effective amount for treatment of the target disease. The recipient organism may be a vertebrate, for example, a mammal, bird, amphibian or reptile and is preferably a mammal. Examples of the mammal include: nonprimates such as dog, cat, horse, pig, cattle, goat, sheep, mouse and rat; and primates such as human, chimpanzee and gorilla. The mammal is preferably a human.

The “pharmaceutically effective amount” in the present specification refers to a dose required for the microvesicle contained in the pharmaceutical composition of the present invention to prevent or treat the target disease or alleviate symptoms with few or no harmful adverse reactions against the recipient organism. A specific dose differs depending on the type of the disease to be prevented and/or treated, the mechanism of action underlying the occurrence of the disease, the dosage form used, information about a subject and an administration route, etc. The range of the pharmaceutically effective amount and a preferred administration route of the pharmaceutical composition that is administered to a human are generally set on the basis of data obtained from cell culture assay and animal experiments. The final dose can be determined and adjusted by the judgment of, for example, a physician, according to an individual subject. The information about the subject to be taken into consideration in that case includes the degree of progression or severity of the disease, general health conditions, age, body weight, sex, diet, drug sensitivity and resistance to treatment, etc. In one embodiment, when the transgene encodes a protein, the pharmaceutical composition of the present invention may be administered in one or more doses of 1×10⁴ to 1×10⁸ transfection unit (t.u.)/kg body weight, for example, 1×10⁵ to 1×10⁷ t.u./kg body weight or 2×10⁵ to 5×10⁶ t.u./kg body weight, per single dose, by direct injection into affected sites or intravenous injection. Here, the transfection unit can be determined by introducing a transgene into a cell (for example a human embryonic kidney 293T cell) using 10 μg of a lentiviral vector of the present invention (for example a DNA vector such as a plasmid vector, e.g., pRBL0213T); determining the amount of the transgene product (e.g., protein, such as PTEN protein) in a fraction of microvesicles (my) released from the cell, e.g., on the basis of ELISA assay; and normalizing the determined amount (transfection efficiency) as one transfection unit being equivalent to 20 pg of the transgene product (e.g., protein) per 1000 cells. The pharmaceutical composition of the present invention may be administered twice or more at predetermined intervals of time, for example, every 1 hour, 3 hours, 6 hours or 12 hours, every day, every 2 days, 3 days or 7 days, or every 1 month, 2 months, 3 months, 6 months or 12 months. Any other parenteral administration or oral administration can be performed in an amount that follows those described above. In the case of particularly severe symptoms, the dose may be increased according to the symptoms.

The administration of the pharmaceutical composition may be systemic administration or local administration and can be appropriately selected according to the type of the disease, the site where the disease occurs, or the degree of progression, etc. If the disease occurs at a local site, the pharmaceutical composition is preferably administered locally by direct administration to the local site (e.g., tumor) and its neighborhood using injection or an indwelling catheter or the like. This is because the microvesicle of the present invention can be administered in a sufficient amount to the site (tissue or organ) to be treated and has no influence on other tissues. Meanwhile, as in metastatic cancer, the site to be treated may not be identified, or the disease may occur systemically. In that case, systemic administration through intravenous injection or the like is preferred. This is because the microvesicle of the present invention can be spread systemically via blood flow, thereby permitting administration to even a lesion that cannot be found by diagnosis.

The pharmaceutical composition can be administered by any appropriate method that does not inactivate the active ingredient contained therein. For example, the administration may be parenteral (e.g., injection, aerosol, application, eye drop, nasal drop or indwelling catheter) or oral. Injection is preferred.

In the case of administration by injection, the injection site may be a non-limiting site where the microvesicle of the present invention can exert its functions and attain the purpose of the pharmaceutical composition. Examples of the injection site include intravenous, intraarterial, intrahepatic, intramuscular, intraarticular, intramedullary, intraspinal, intraventricular, percutaneous, subcutaneous, intracutaneous, intraperitoneal, intranasal, intestinal and sublingual sites. In one embodiment, direct administration to tumor is also preferred.

The pharmaceutical composition of the present invention can be used to effectively achieve the prevention and/or treatment of the disease by the transgene product or the like contained in the microvesicle.

Thus, the present invention also provides a method for treating a patient, comprising administering the microvesicle produced by the method of the present invention to the patient in need of introduction of the transgene or the transgene product. The patient may suffer from cancer, diabetes, neurodegenerative disease, immune dysfunction, inflammation, liver cirrhosis, arteriosclerosis, thrombus or infection. Preferably, the patient suffers from cancer. More specifically, the cancer may be selected from the group consisting of, for example, colon cancer, pancreatic cancer, kidney cancer, lung cancer, neuroblastoma, breast cancer, ovarian cancer, gastric cancer, prostate cancer, thyroid cancer and malignant lymphoma. In one embodiment, the transgene product contained in the microvesicle may cause a reduced expression of CDC6. For example, the transgene product may be a shRNA targeting CDC6. In that case, the disease to be treated, for example, cancer, may involve an elevated expression of CDC6. Administration methods and administration sites for the microvesicle to the patient can be used as described above in relation to the administration methods and administration sites for the pharmaceutical composition.

The treatment method of the present invention can effectively treat the disease such as cancer in the patient. In one preferred embodiment, the method for treatment of cancer of the present invention can inhibit (reduce) the growth of tumors.

### 6. Description of Sequence

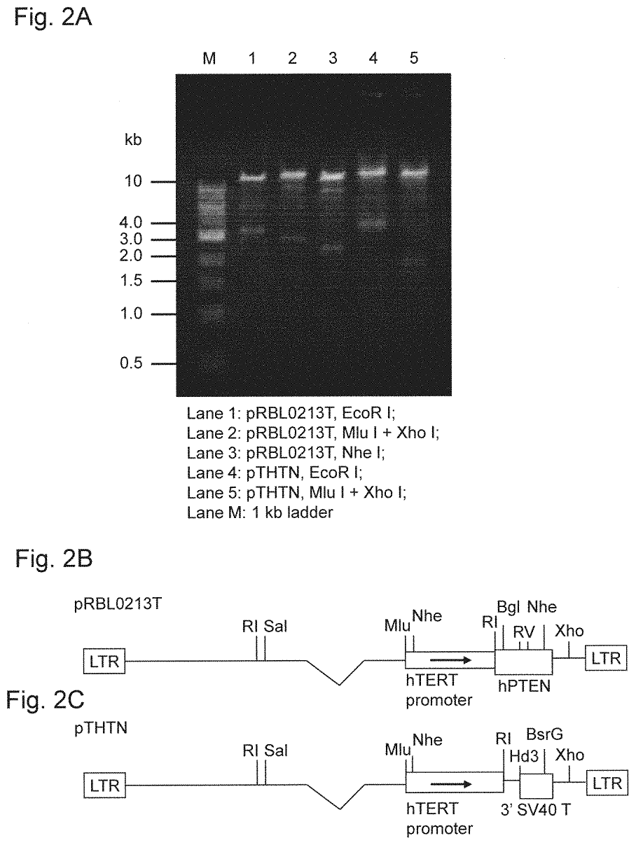

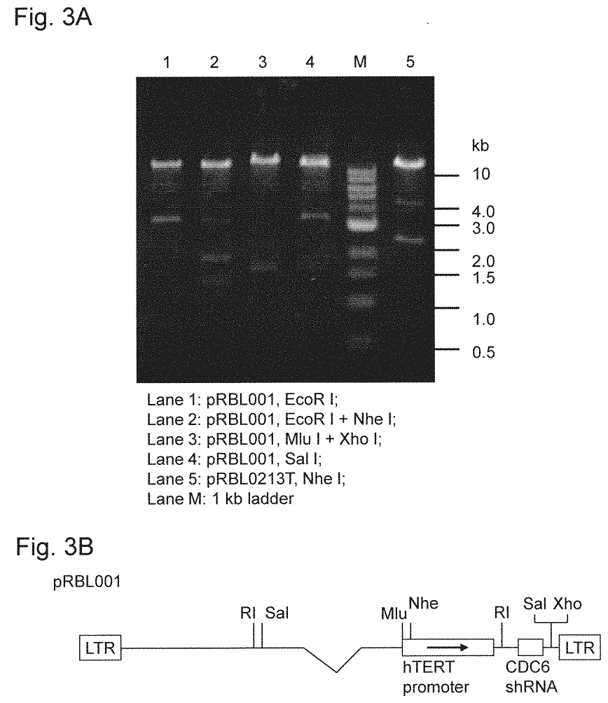

SEQ ID NO: 1 shows the nucleotide sequence of the telomerase reverse transcriptase (TERT) gene promoter from Homo sapiens, which was used to produce plasmids pRBL0213T, pTHTN and pRBL001 (see, FIGS. 2A and 2B and FIG. 3B). The genomic DNA sequence comprising human TERT gene is available under GenBank accession No. AF128893.

SEQ ID NO: 2 shows the nucleotide sequence of the 5′ portion of TERT transcribed region from Homo sapiens, which was inserted to produce plasmids pRBL0213T, pTHTN and pRBL001.

SEQ ID NO: 3 shows the nucleotide sequence comprising the TERT gene promoter (SEQ ID NO: 1) and the 5′ portion (SEQ ID NO: 2) of TERT transcribed region from Homo sapiens, which was used to produce plasmids pRBL0213T, pTHTN and pRBL001. SEQ ID NO: 3 comprises upstream sequence, the whole first exon and a part of the second exon of the TERT gene. The first exon starts at position 1390 and ends at position 1670 of SEQ ID NO: 3. The second exon starts at position 1775 of SEQ ID NO: 3.

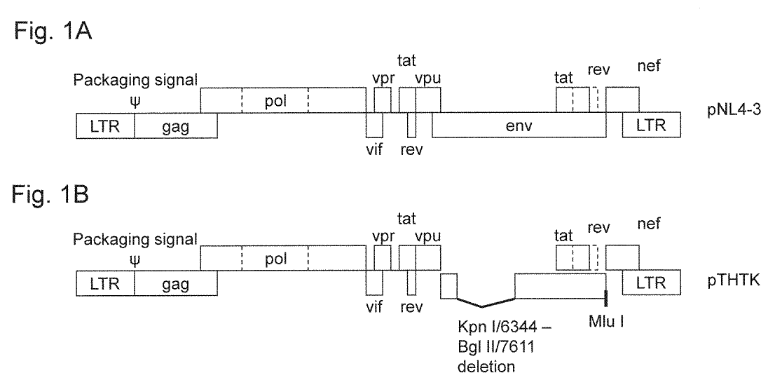

SEQ ID NO: 4 shows the sequence of recombinant plasmid pNL4-3 clone comprising the nucleotide sequence of full-length genomic DNA of HIV-1 NL4-3 strain (GenBank accession No. M19921). The nucleotide sequence from position 1 to position 9709 of SEQ ID NO: 4 corresponds to HIV-1 genome (5′ LTR to 3′ LTR). The nucleotide sequence from position 6221 to position 8785 of SEQ ID NO: 4 encodes env protein. For the structure of HIV-1 genome, see FIG. 1A.

SEQ ID NO: 5 shows the nucleotide sequence of HIV-1 genomic region in plasmid pTHTK, i.e., the nucleotide sequence from 5′ LTR to 3′ LTR in pTHTK (see FIG. 1B). The nucleotide sequence from position 6344 to position 7611 of SEQ ID NO: 4 was deleted from pNL4-3. Further, pNL4-3 was cleaved between positions 8650 and 8651 of SEQ ID NO: 4 with restriction enzyme Hpa I and the nucleotide sequence from position 7383 to position 7674 of SEQ ID NO: 5 was inserted thereinto to produce pTHTK.

SEQ ID NO: 6 shows the nucleotide sequence of PTEN CDS (from start codon to stop codon) from Homo sapiens, which was used to produce plasmid pRBL0213T (see FIG. 2B). Human PTEN mRNA sequence is available under GenBank accession No. N. Mex. 000314.

SEQ ID NO: 7 shows the nucleotide sequence of CDC6 CDS from Homo sapiens. Human CDC6 mRNA sequence is available under GenBank accession No. N. Mex. 001254.

SEQ ID NOs: 8 and 9 show the oligonucleotide sequences Cdc6-5-A and Cdc6-3-A, respectively, which were used to prepare double-stranded oligonucleotide inserted into plasmid pRBL001 (see FIG. 3B).

SEQ ID NO: 10 shows the DNA sequence encoding CDC6 shRNA.

SEQ ID NO: 11 shows the nucleotide sequence of primer B-NLR8950.

SEQ ID NO: 12 shows the nucleotide sequence of oligonucleotide HD-A.

SEQ ID NO: 13 shows the nucleotide sequence of oligonucleotide HD-S.

SEQ ID NO: 14 shows the nucleotide sequence of forward primer BHU5-S2.

SEQ ID NO: 15 shows the nucleotide sequence of reverse primer HDA/SBOT.

SEQ ID NO: 16 shows the nucleotide sequence of primer 5′ LTRU5.

SEQ ID NO: 17 shows the nucleotide sequence of primer 3′ NL5850.

SEQ ID NO: 18 shows the nucleotide sequence of primer 3′ NL8960.

SEQ ID NO: 19 shows the nucleotide sequence of the antisense sequence of CDC6 shRNA.

SEQ ID NO: 20 shows the nucleotide sequence of the linker in CDC6 shRNA.

SEQ ID NO: 21 shows the nucleotide sequence of the partial fragment of SV40 large T antigen gene.

SEQ ID NO: 22 shows the amino acid sequence of the human PTEN protein encoded by the PTEN gene of SEQ ID NO: 6.

SEQ ID NO: 23 shows the nucleotide sequence (CDS) of human p16^{INK4a} gene (GenBank accession No. L27211).

SEQ ID NO: 24 shows the amino acid sequence of the human p16^{INK4a} protein encoded by the p16^{INK4a} gene of SEQ ID NO: 23.

SEQ ID NO: 25 shows the nucleotide sequence of plasmid pCMV p16INK4a.

SEQ ID NO: 26 shows the nucleotide sequence (CDS) of human p53 gene (GenBank accession No. BC003596).

SEQ ID NO: 27 shows the amino acid sequence of the human p53 protein encoded by the p53 gene of SEQ ID NO: 26.

### EXAMPLES

Hereinafter, the present invention will be described more specifically with reference to Examples. However, the technical scope of the present invention is not limited to these Examples.

### Example 1 Recombinant Lentiviral Plasmid Vector

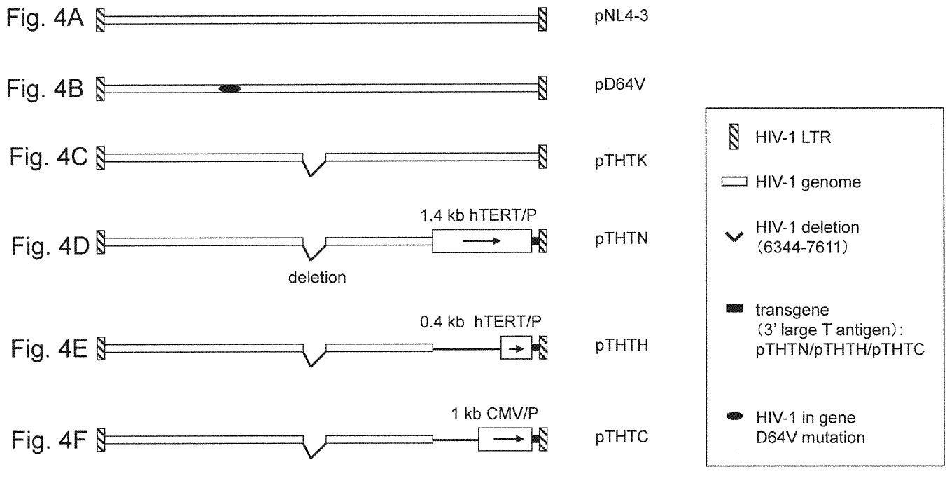

Recombinant lentiviral plasmid vectors used in Examples described later are derived from HIV-1 genomic DNA vector pNL4-3 (FIGS. 1A and 4A) (Adachi A et al., J. Virol., 1986, p. 284-291). Recombinant pNL4-3 clone sequence comprising the full-length genomic DNA sequence of HIV-1 NL4-3 strain is available under GenBank accession No. M19921 (SEQ ID NO: 4). A recombinant lentiviral plasmid vector pD64V (FIG. 4B) has a mutation that results in the substitution of aspartic acid at position 64 of HIV-1 integrase with valine in pNL4-3. The pD64V was kindly provided by Dr. Sam Chow, the Department of Molecular and Medical Pharmacology of University of California Los Angeles (UCLA).

Other recombinant lentiviral plasmid vectors used were prepared from construct pTHTK (FIG. 1B). The pTHTK is derived from the pNL4-3 described above (FIGS. 1A and 1B). The pTHTK lacks nucleotides 6344 to 7611 (based on SEQ ID NO: 4; the same holds true for the description below unless otherwise specified) in the HIV-1 genomic DNA of pNL4-3 as a result of treatment with restriction enzymes Kpn I and Bgl II. Specifically, two sites, Kpn I (positions 6343 to 6348) and Bgl II (positions 7611 to 7616), in HIV-1 backbone were blunt-ended and re-ligated to prepare pTHTK deficient in HIV-1 env gene encoding envelope p120 glycoprotein. Further, the pTHTK was cleaved at the only one Hpa I site (positions 8648 to 8653) and modified by the addition of Mlu I linker (nucleotide sequence from positions 7383 to 7674 of SEQ ID NO: 5). The nucleotide sequence of the HIV-1 genomic region (i.e., 5′ LTR to 3′ LTR) of pTHTK is shown in SEQ ID NO: 5.

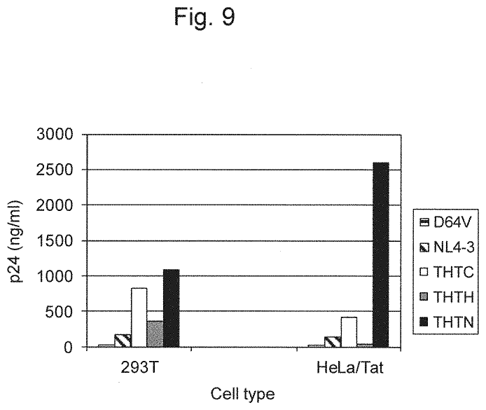

For ligation with a DNA fragment (transgene expression cassette) to be cloned, the vector pTHTK was cleaved 5′-terminally at Mlu I site and 3′-terminally at Xho I site (positions 8887 to 8892). The insertion of the transgene expression cassette to the Mlu I-Xho I site of this vector disrupts HIV-1 nef gene open reading frame. Nef protein encoded by the nef gene plays a role in viral infection and spreading and extracellular release of virion. Therefore, the insert-containing (i.e., nef-defective) lentiviral plasmid vector will produce viral particles less infectious for CD4⁺ cells, unlike wild-type HIV-1 NL4-3 strain. In contrast, for VSV/G pseudotyped RBL0213T, RBL001, THTN and so on, whether Nef defective or not, more viral particles were budding out than that of wild-type NL4-3, as described below (see, e.g., FIG. 9).

The pTHTK backbone thus obtained was used to construct recombinant lentiviral plasmid vectors as described below.

(a) pRBL0213T

The pRBL0213T is a plasmid in which DNA encoding human PTEN gene was inserted under control of hTERT gene promoter (hTERT promoter) (FIG. 2B).

Plasmid pGL-1375 was used as a source of the hTERT promoter (Takakura et al., Cancer Res., 1999, p. 551-557). The pGL-1375 was kindly provided by Dr. Satoru Kyo (Kanazawa University of Japan). The pGL-1375 has hTERT promoter DNA fragment inserted between Mlu I and Bgl II sites. hTERT 5′ upstream sequence is available under GenBank accession No. AF128893. The hTERT promoter DNA fragment was excised from pGL-1375 and inserted to the pTHTK backbone.

Human PTEN mRNA sequence is available under GenBank accession No. N. Mex._000314 (SEQ ID NO: 6). The PTEN gene-encoding DNA used to construct pRBL0213T was excised from vector pcDNA3.1/CMV-hPTEN (kindly provided by Dr. Hong Wu, Department of Molecular Medicine of University of California Los Angeles (UCLA)). The PTEN gene-encoding DNA was further inserted to the pTHTK backbone to prepare pRBL0213T.

The pRBL0213T comprises: the nucleotide sequence of SEQ ID NO: 3 consisting of the hTERT promoter sequence (SEQ ID NO: 1) and the 5′ portion (SEQ ID NO: 2) of hTERT transcribed region containing the first exon and a part of the second exon of the hTERT gene; and the PTEN coding sequence.

(b) pTHTN

The pTHTN is a plasmid in which a partial fragment of SV40 large T antigen gene was inserted under control of hTERT promoter (FIG. 2C).

The partial fragment (SEQ ID NO: 21) of the SV40 large T antigen gene was inserted downstream of the hTERT promoter inserted in the pTHTK backbone to prepare pTHTN. Simian virus 40 genome sequence comprising the SV40 large T antigen gene is available under GenBank accession No. NC_001669. The pTHTN comprises: the nucleotide sequence of SEQ ID NO: 3 consisting of the hTERT promoter sequence (SEQ ID NO: 1) and the 5′ portion (SEQ ID NO: 2) of hTERT transcribed region containing the first exon and a part of the second exon of the hTERT gene; and the partial fragment (SEQ ID NO: 21; from nucleotide 5175 (Hind III) to nucleotide 4863 (Hae III) of DNA sequence of GenBank accession number: NC_001669) of the SV40 large T antigen gene.

(c) pRBL001

The pRBL001 is a plasmid (plasmid for producing recombinant lentiviral particle THTD) in which DNA encoding CDC6 shRNA was inserted under control of hTERT promoter (FIG. 3B).

The sequences of synthetic oligonucleotides used to construct the DNA encoding CDC6 shRNA are shown below.

Cdc6-5-A:
(SEQ ID NO: 8)
5′-GATCCCCAGGCACTTGCTACCAGCAATTCAAGAGATTGCTGGTA
GCAAGTGCCTTTTTTGGAAA-3′
Cdc6-3-A:
(SEQ ID NO: 9)
5′-AGCTTTTCCAAAAAAGGCACTTGCTACCAGCAATCTCTTGAATT
GCTGGTAGCAAGTGCCTGGG-3′

For construction of pRBL001, the synthetic oligonucleotides Cdc6-5-A and Cdc6-3-A were mixed in equimolar amounts, denatured, and re-annealed to prepare a double-stranded oligonucleotide. This double-stranded oligonucleotide was blunt-ended and then inserted to the EcoR V site of subcloning vector pBlueScript. DNA sequencing was performed to confirm that the double-stranded CDC6 oligonucleotide was inserted in the correct orientation to have the correct sequence. The resulting subclone was linearized at Xba I site, blunt-ended, and modified by the ligation of the Mlu I linker described above. A DNA fragment comprising the hTERT promoter sequence was obtained by the digestion of the pGL3-1375 described above with Mlu I and Bgl II and cloned into the Mlu I and BamH I sites of the subcloning vector carrying the double-stranded CDC6 oligonucleotide. A DNA fragment containing the hTERT promoter and the double-stranded CDC6 oligonucleotide was excised from the resulting subclone by Mlu I and Xho I double digestion. The resulting fragment was purified and inserted to the pTHTK backbone that was cleaved with Mlu I and Xho I and gel-purified, to prepare pRBL001.

The pRBL001 comprises: the nucleotide sequence of SEQ ID NO: 3 consisting of the hTERT promoter sequence (SEQ ID NO: 1) and the 5′ portion (SEQ ID NO: 2) of hTERT transcribed region containing the first exon and a part of the second exon of the hTERT gene; and the CDC6 shRNA-encoding sequence (SEQ ID NO: 10). The CDC6 shRNA comprises: a CDC6 shRNA antisense sequence (SEQ ID NO: 19); a linker (SEQ ID NO: 20); and a sense sequence consisting of a sequence complementary to the antisense sequence and a 3′ poly U overhang of 5 bases.

Human CDC6 mRNA-targeting shRNA (CDC6 shRNA) produced via the transcription of pRBL001 can specifically cause CDC6 mRNA degradation via RNA interference (RNAi) to result in the knockdown of DNA replication initiator CDC6 protein. Human CDC6 mRNA sequence is available under GenBank accession No. N. Mex._001254. The nucleotide sequence of human CDC6 CDS is shown in SEQ ID NO: 7. A guide strand from the CDC6 shRNA antisense sequence of SEQ ID NO: 19 binds to CDC6 mRNA to cause RNAi.

(d) pTHTH

pTHTH is a plasmid in which a partial fragment of SV40 large T antigen gene was inserted under control of hTERT promoter that lacks 5′ portion (1 kb) (FIG. 4E).

To construct pTHTH, the plasmid pGL-378 (kindly provided by Dr. Satoru Kyo, Kanazawa University, Japan) carrying the shortened hTERT promoter sequence with the 5′ deletion (1 kb) and luciferase gene was cleaved with Mlu I plus Hind III to obtain a DNA fragment comprising the shortened hTERT promoter sequence that lacks the 5′ portion (1 kb). The DNA fragment was purified and incorporated into the plasmid pBluescript at the Mlu I and Hind III sites to generate plasmid pEND-HTPs. The partial fragment (SEQ ID NO: 21) of SV40 large T antigen gene was excised from a plasmid carrying the SV40 genome DNA with Hind III and Hae III and inserted into pEND-HTPs at the Hind III and Hinc II sites. The resulting plasmid subclone was then cleaved with Mlu I and Xho I, and the excised DNA fragment comprising the shortened hTERT promoter sequence that lacks the 5′ portion (1 kb) and the partial fragment of SV40 large T antigen gene was purified and inserted into pTHTK at Mlu I and Xho I sites to generate pTHTH.

The pTHTH comprises: a nucleotide sequence consisting of the hTERT promoter sequence lacking the 5′ portion (1 kb) and the 5′ portion (SEQ ID NO: 2) of hTERT transcribed region containing the first exon and a part of the second exon of the hTERT gene; and the partial fragment (SEQ ID NO: 21) of the SV40 large T antigen gene.

(e) pTHTC

The pTHTC is a plasmid in which a partial fragment of SV40 large T antigen gene was inserted under control of a human cytomegalovirus (CMV) promoter (FIG. 4F).

The CMV promoter was inserted to the pTHTK backbone. The partial fragment (SEQ ID NO: 21) of the SV40 large T antigen gene was inserted downstream of the CMV promoter in the pTHTK to prepare pTHTC.

### Example 2 Preparation of Pseudotyped Recombinant Lentiviral Particle

The recombinant lentiviral plasmid vectors deficient in HIV-1 env gene described in Example 1 cannot produce infectious viral particles in themselves. Thus, in this Example, recombinant lentiviral particles (pseudotyped) were prepared via the cotransfection of human embryonic kidney 293T cells with each recombinant lentiviral plasmid vector described in Example 1 and plasmid pCMV-VSV/G expressing the envelope glycoprotein G of vesicular stomatitis virus (VSV). The pCMV-VSV/G was kindly provided by Dr. Sam Chow, the Department of Molecular and Medical Pharmacology of University of California Los Angeles (UCLA). Specific experimental procedures are as described below.

### 1. Plasmid DNA Transfection

15 ml of DMEM/high-glucose (Hyclone, Utah, USA) complete medium (supplemented with 10% fetal bovine serum, 100 units/ml penicillin and 100 μg/ml streptomycin (Hyclone, Utah, USA)) was added to T75 flask, to which human embryonic kidney 293T cells were then inoculated. The 293T cells were proliferated in 5% CO₂ incubator at 37° C. and increased to cultures in ten T75 flasks while subcultured.

2×HEPBS (100 ml) was prepared as follows: 1 g of Hepes (acidic salt), 1.6 g of NaCl, 0.75 ml of Na₂HPO₄ (0.25 M) and 1 ml of KCl (1 M) were dissolved in an appropriate amount of ddH₂O. The pH of the solution was adjusted to 6.9 using NaOH (5 M) and then finely adjusted to 7.12 to 7.14 using NaOH (1 M). ddH₂O was added to this solution up to 100 ml in total and the resulting solution was passed through a syringe filter with 0.22 μm pore size to prepare 2×HEPBS.

The followings were added to 50-ml tube:
(a) 500 μl of plasmid DNA solution containing 170 μg of each recombinant lentiviral plasmid vector deficient in HIV-1 env gene described in Example and 30 μg of pCMV-VSV/G,
(b) 1,650 μl of ddH₂O, and
(c) 350 μl of 2 M CaCl₂.

The solution was gently mixed and then 2,500 μl of 2×HEPBS was added dropwise thereto with gentle stirring to circumvent the formation of large precipitates, thereby preparing a transfection mixture.

The tube containing the transfection mixture was left at room temperature for 20 minutes. The medium was discarded from the ten T75 flasks in which the 293T cells were proliferated. Then, 12 ml of fresh DMEM/high-glucose complete medium was added to each flask. 500 μl of the transfection mixture was gradually added to each flask. The cells were incubated in 5% CO₂ incubator at 37° C. for 8 hours.

### 2. Cell Culture and Collection of Medium

The medium was discarded and 15 ml of fresh DMEM/high-glucose complete medium was added to each flask. The cells were incubated in 5% CO₂ incubator at 37° C. for 36 hours and the medium (medium after 36 hours) was collected. 15 ml of fresh DMEM/high-glucose complete medium was added to each flask. The cells were incubated in 5% CO₂ incubator at 37° C. for additional 36 hours and the medium (medium after 72 hours) was collected. 15 ml of fresh DMEM/high-glucose complete medium was added to each flask. The cells were further incubated in 5% CO₂ incubator at 37° C. for 24 hours and the medium (medium after 96 hours) was collected. Each 50-ml tube containing the medium collected was centrifuged at 3,000 rpm and 4° C. for 5 minutes.

### 3. Titeration

200 μl of the medium supernatant was transferred to another tube and diluted 50- to 200-fold by the addition of 1×PBS. This medium supernatant diluted was assayed for the amount of virus-derived protein p24 released into the medium from the cells infected by recombinant lentiviral particles using HIV-1 p24 antigen ELISA assay kit (Coulter Inc., Miami, Fla., USA), thereby determine the titers of the viral particles.

### 4. Purification

The medium supernatant collected by centrifugation in “2. Cell culture and collection of medium” described above was transferred to 250-ml high-speed centrifuge tube and centrifuged at 9,000×g and 4° C. for 60 minutes. The resulting supernatant was concentrated 5-fold by ultrafiltration using Vivaspin® 20 (1,000 kDa molecular weight cutoff (mw. co.)) (Sartorius, NY, USA). MMP solution was added to the resulting solution and viral particles were precipitated overnight at 4° C. The MMP solution (300 ml) was prepared by: dissolving 90 g of PEG 8,000 (molecular biological grade) and 30 ml of NaCl (UltraPure) (5 M) in an appropriate amount of ddH₂O; and adding ddH₂O to the solution up to 300 ml in total.

The solution in which viral particles were precipitated was centrifuged at 9,000×g and 4° C. for 30 minutes to form pellet containing the viral particles. The supernatant was discarded and the pellet was resuspended in 10 ml of 1×PBS to obtain a solution containing the viral particles.

The viral particles in an aliquot of the solution were PEGylated (Croyle et al., J. Virol., 2004, Vol. 78, p. 912-921). The PEGylation was performed by adding 0.4 ml of PEGylation solution (33 mg/mL methoxy PEG succinimidyl carbonate NHS (mPEG-NHS, m.w. 10K (NANOCS, USA)), 30 mM HEPES-KOH, pH 7.5, 500 mM NaCl) to approximately 10 ml of the solution containing the viral particles and incubating the mixture at room temperature for 60 minutes on a rotary platform.

The solution containing the viral particles PEGylated or unPEGylated was dialyzed against 1×PBS at 4° C. using Slide-A-Lyzer cassette (20 kDa mw. co.) (Thermo Scientific, IL, USA) and 1×PBS was replaced with fresh one every 24 hours for 2 days. The viral particle solution thus dialyzed was concentrated 30-fold using AmiconUltra 15 (100 kDa mw. co.) (Millipore, MA, USA). This viral particle solution concentrated was passed through a syringe filter with 0.45 μm pore size and the resulting preparation was stored at −80° C.

In this way, the pseudotyped recombinant lentiviral particles were successfully prepared. The pseudotyped recombinant lentiviral particles prepared from the recombinant lentiviral plasmid vectors pTHTK, pRBL0213T, pTHTN, pRBL001, pTHTH and pTHTC described in Example 1 are referred to as THTK, RBL0213T, THTN, THTD, THTH and THTC, respectively, in subsequent Examples.

The recombinant lentiviral particles thus prepared can infect various human proliferating cells and non-dividing and dividing cells as described later. Target cells infected by the recombinant lentiviral particles synthesize lentiviral DNA, which is in turn integrated into host chromosomal DNA. Thus, the resulting recombinant lentiviral particles can be used as lentiviral vectors.

### Example 3 Study on Ability of Recombinant Lentiviral Particle to Produce Infectious Viral Particle

On the basis of the detection of LTR-Tag formed by ligation-mediated PCR (LM-PCR), the pTHTK-derived pseudotyped recombinant viral particle THTK was examined for its ability to produce infectious viral particles after infection of cells.

1. Preparation of Bpm I Site in pTHTK

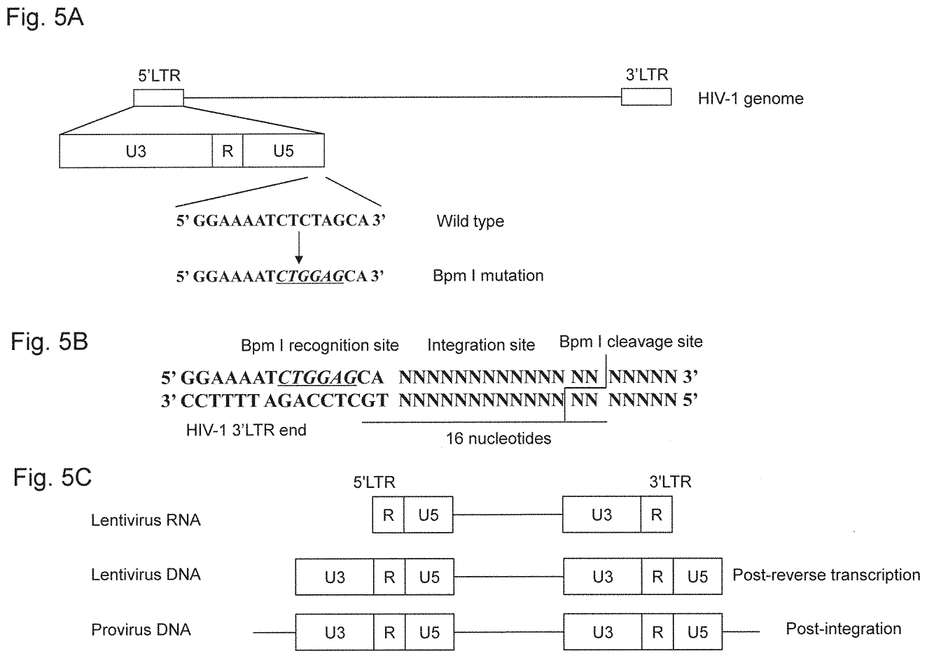

In order to carry out LM-PCR, pTHTK was first modified to prepare new Bpm I site in HIV-1 genome (FIG. 5A). HIV-1 5′ LTR region has three elements (U3, R and U5). The Bpm I site was prepared via overlap PCR at the 3′ end of the U5 element of 5′ LTR in pTHTK. Bpm I is a class II S restriction enzyme. Bpm I recognizes 5′-CTGGAG-3′. Bpm I cleaves DNA at 16 nucleotides 3′ of its recognition site in a strand comprising this 5′-CTGGAG-3′ and at 14 nucleotides 5′ of its recognition site in another strand to produce a 3′ overhang of 2 nucleotides (FIG. 5B).

FIG. 5C shows the terminal structure of lentiviral genome. Lentiviral RNA is produced by transcription from the 5′LTR R element to the 3′ LTR R element of proviral DNA. Accordingly, the lentiviral RNA has neither the 5′ LTR U3 element nor the 3′ LTR U5 element at the ends. However, when the lentiviral RNA is reverse transcribed into lentiviral DNA, the 3′ LTR U3 element and the 5′ LTR U5 element are copied to the 5′ LTR U3 element and the 3′ LTR U5 element, respectively. Then, this lentiviral DNA is integrated into host genome to form proviral DNA.

Thus, the new Bpm I site in the 5′ LTR U5 element produced in the HIV-1 genome of pTHTK is copied to the 3′ LTR U5 element when lentiviral DNA is synthesized in infected cells. This new Bpm I site has no influence on THTK virion assembly, viral particle infection and proviral DNA integration in infected cells. Host cells are infected by the viral particle THTK derived from pTHTK having this new Bpm I site so that proviral DNA having the Bpm I site copied at a position distant by 2 nucleotides (5′-CA-3′) from the end in the 3′ LTR U5 element is integrated into host genome. Thus, host genomic DNA from the infected cells is cleaved at a position distant by 14 nucleotides from the integration site of the proviral DNA as a result of Bpm I treatment (FIG. 5B).

### 2. Preparation of LTR-Tag

Human uterine cervix cancer HeLa cells expressing HIV-1 Tat protein (HeLa/tat cells) were infected by coincubation with the viral particle THTK prepared as described in Example 2 from the pTHTK having the new Bpm I site described above. The HeLa/tat cells were kindly provided by Dr. Sam Chow, the Department of Molecular and Medical Pharmacology of University of California Los Angeles (UCLA). 3, 5, 10 and 15 days after the infection, host genomic DNA was isolated from the HeLa/tat cells. The genomic DNA was subjected to Kas I and Xho I double digestion. Each of Kas I (positions 637 to 642; based on SEQ ID NO: 4; the same holds true for the description below) and Xho I (positions 8887 to 8892) is a unique site in HIV-1 genome. The Kas I site is located 4 nucleotides downstream of 5′ LTR and the Xho I site is located approximately 190 nucleotides upstream of 3′ LTR. The Kas I and Xho I double digestion separates proviral DNA from the host genomic DNA while the 5′ LTR and 3′ LTR sequences of the proviral DNA remain with the host genomic DNA. Further, the Kas I recognition site is located between the Bpm I recognition site (positions 627 to 632) prepared in 5′ LTR and the Bpm I cleavage site at 16 nucleotides downstream thereof. Therefore, even Kas I digestion followed by Bpm I digestion does not result in Bpm I digestion based on the Bpm I site in 5′ LTR.

Next, special DNA extension reaction shown below was performed. A primer derived from a sequence of approximately 100 nucleotides upstream of 3′ LTR is used in this reaction. Therefore, the 3′ LTR of the proviral DNA and the host genomic DNA downstream thereof are amplified markedly while the ratio of contaminating DNA, specifically, DNA derived from the more upstream proviral DNA drastically decreases. The nucleotide sequence of the primer B-NLR8950 for DNA extension is shown below.

B-NLR8950:
(SEQ ID NO: 11)
5′-B-GTGCCTGGCTAGAAGCACAAG-3′

This sequence has biotin-labeled nucleotides and is derived from a sequence from positions 8950 to 8970 in the genomic DNA of the HIV-1 NL4-3 strain. This primer was used to perform the DNA extension reaction as shown below.

DNA extension reaction solution:

| | | |
|---|---|---|
| B-NLR8950 | 50 | μm |
| Kas I + Xho I-digested host genomic DNA | 10 | μg |
| dNTPs | 200 | μm |
| Taq DNA polymerase | 5 | units |

The tube containing the DNA extension reaction solution described above was incubated at 94° C. for 5 minutes, at 55° C. for 5 minutes and at 72° C. for 30 minutes for DNA extension reaction to obtain biotinylated DNA.

This biotinylated DNA was purified as follows via binding to streptavidin-magnetic beads (Dynabeads M-280): approximately 5 pmol of the biotinylated DNA was mixed with 40 pmol of Dynabeads M-280 in a tube and the mixture was incubated at room temperature for 30 minutes. The tube was placed on a magnetic stand (Dynal MPC stand) and left for 2 minutes. Then, the beads were washed with TE (pH 8.0) buffer. The washing of the beads was performed using 1 ml of TE and further repeated two times. The beads were centrifuged for 2 seconds and the supernatant was discarded to purify the biotinylated DNA.

Then, the biotinylated DNA was subjected to Bpm I digestion. The biotinylated DNA was digested with Bpm I overnight at 37° C. The biotinylated DNA digested was purified via Dynabeads M-280 and then ligated with a double-stranded oligo linker. One end of the double-stranded oligo linker has a 3′ overhang of 2 nucleotides and is complementary to the Bpm I-digested end. The other end of the double-stranded oligo linker has a 5′ overhang of 3 nucleotides and efficiently prevents the self-ligation of the linker. The nucleotide sequences of oligonucleotides HD-A and HD-S(provided by Dr. Sam Chow, University of California, UCLA) constituting the double-stranded linker are shown below.

HD-A:
(SEQ ID NO: 12)
5′-CACGCGTCGCATCATATCTCCAGGTGTGACAG-3′
HD-S:
(SEQ ID NO: 13)
5′-CCTCTGTCACACCTGGAGATATGATGCGACGCGTGNN-3′

The 3′-terminal “NN” of HD-S represents a degenerate sequence indicated by any combination of “A”, “T”, “G” and “C.”

The ligation of the biotinylated DNA with the double-stranded oligo linker was performed at room temperature for 16 hours. The biotinylated DNA linked to the double-stranded oligo linker was purified via Dynabeads M-280.

### 3. Detection of LTR-Tag

In order to detect LTR-Tag, PCR amplification was performed. The following primers were used in the PCR reaction:

Forward primer BHU5-S2:
(SEQ ID NO: 14)
5′-GAGTGCTCAAAGTAGTGGT-3′
Reverse primer HDA/SBOT:
(SEQ ID NO: 15)
5′-CTGTCACACCTGGAGATATGAT-3′

The nucleotide sequences of the forward primer and the reverse primer are derived from positions 9617 to 9636 of the HIV-1 genomic DNA and one strand HD-S of the double-stranded oligo linker, respectively.

The following PCR reaction was performed: 1 cycle involving 94° C. for 4 minutes, and 30 cycles each involving 94° C. for 60 seconds, 60° C. for 30 seconds and 72° C. for 90 seconds, followed by 72° C. for 10 minutes and 4° C. for 1 to 12 hours. The biotinylated DNA linked to the double-stranded oligo linker described above was used as template DNA in the PCR reaction.

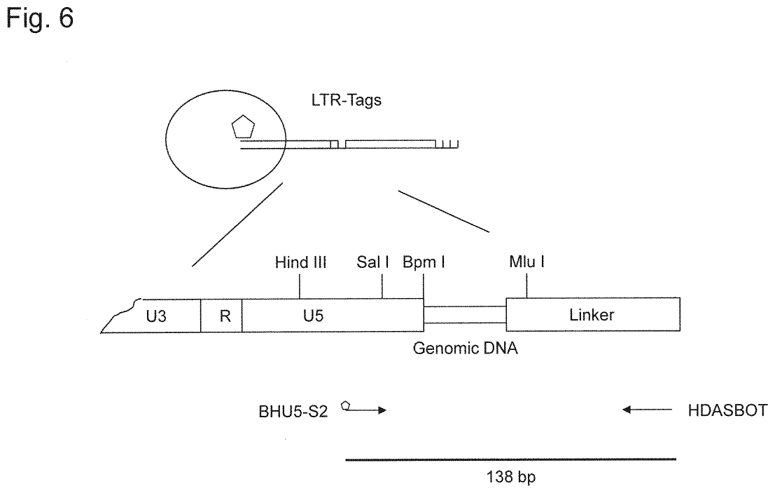

After PCR, 5 μl of each reaction product was loaded onto a gel (2.0% agarose) and electrophoresed in 1×TAE. Approximately 138-bp PCR product (LTR-Tag) was detected provided that the proviral DNA was integrated into the host cell genome (FIG. 6).

### 4. Results

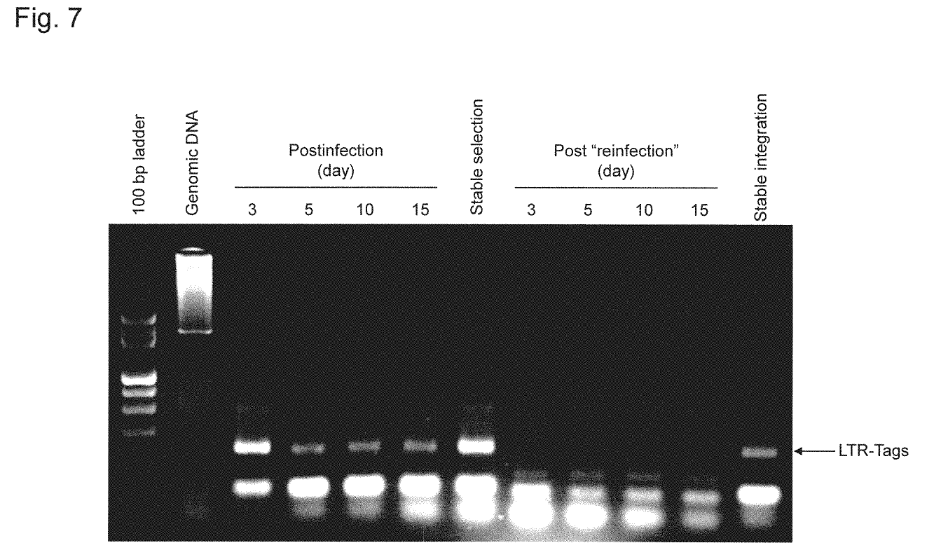

The results are shown in FIG. 7. The detected LTR-Tag (LTR-Tag positive) indicates that the proviral DNA was integrated into the host cell genome. This means that the host cells were infected by the virus. The pseudotyped recombinant lentiviral particle THTK exhibited LTR-Tag positive and was shown to successfully infect the cells (FIG. 7, After infection).

Next, the culture medium of the infected cells was collected, filtered through a filter with 0.45 μm pore size, and added to fresh cultured cells (“reinfection”). Genomic DNA was isolated from these cells and examined for LTR-Tag. As a result, the viral particle exhibited LTR-Tag negative, indicating that the cells were not infected by the viral particle (FIG. 7, After “reinfection”). These results demonstrated that the cells infected by the recombinant lentiviral particle THTK did not produce infectious viral particles. LM-PCR to test whether the recombinant lentiviral particle was unable to replicate in the infected cells was accurate and ended in perfect reproducibility without false positive results, i.e., contamination by the direct PCR of the HIV-1 sequence.

The results described above demonstrated that the pseudotyped recombinant lentiviral particle THTK can infect cells, but the infected cells do not produce infectious viral particles. This indicates that the method for producing microvesicles according to the present invention using the pseudotyped recombinant lentiviral particle as the lentiviral vector does not produce infectious viral particles.

### Example 4 Direct Injection of Recombinant Lentiviral Particle THTD to Human Tumor Transplanted in Nude Mouse

An animal experiment was conducted to directly inject the pseudotyped recombinant lentiviral particle THTD prepared as described in Example 2 to human breast cancer Bcap-37 tumor transplanted in nude mouse. THTD (protein content: 275 μg) was administered by injection to a plurality of sites in the tumor lesion of the animal twice a week for 3 weeks. 48 hours after the final administration, the animal was euthanized by cervical dislocation. For each animal in a control group, 1×PBS was injected to the tumor lesion. The length and width of each tumor were measured twice a week using a standard caliper after the injection. After the euthanasia of the animal by cervical dislocation, its tumor was isolated, measured, and treated for pathological examination.

The proliferation of the THTD-treated tumor was inhibited by 34.70% relative to the control group and the average weight of the tumor was significantly lower than that of the control (p<0.02). The injection of THTD developed strong fibrosis in tumor tissue, thereby suppressing tumor proliferation.

The results described above demonstrated that the pseudotyped recombinant lentiviral particle THTD infects mouse cells and then CDC6 shRNA encoded by the viral genome was produced in the cells to suppress the expression of CDC6, resulting in the suppressed proliferation of tumor. This indicates the possibility that the microvesicle of the present invention comprising CDC6 shRNA also has tumor proliferation suppressive effect.

### Example 5 Enhancement of Viral Genomic RNA Splicing by hTERT Promoter

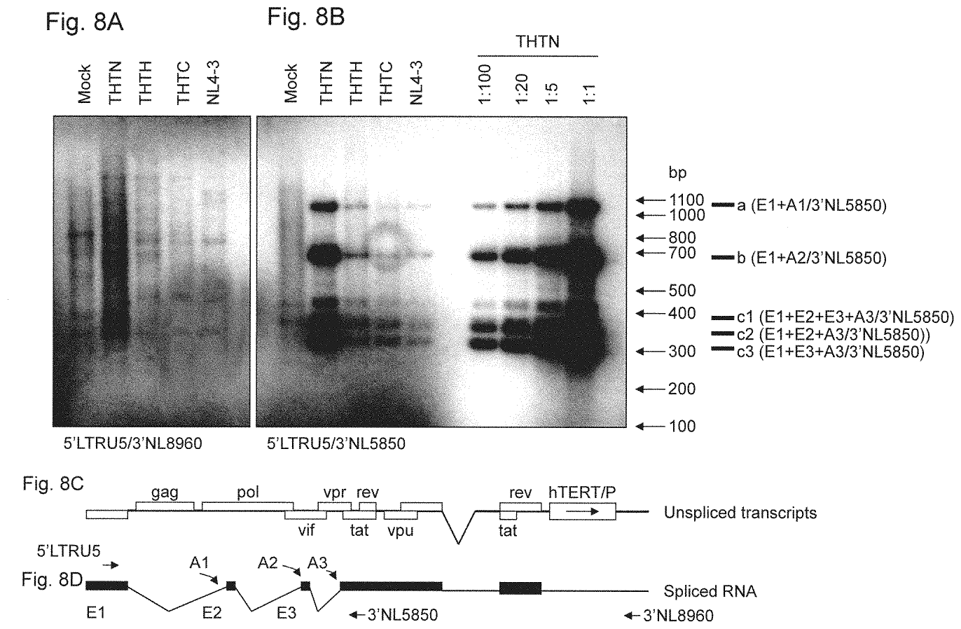

The gene transcription and gene transduction activities of the recombinant viral particle THTN prepared as described in Example 2 were compared with those of other recombinant lentiviral particles and examined by detecting RNA splicing activity by RT-PCR (FIG. 8).

### 1. Viral Infection and Collection of Cell

Human uterine cervix cancer HeLa cells expressing HIV-1 Tat protein (HeLa/tat cells) were inoculated at a concentration of approximately 2×10⁵ cells/well into a 6-well plate and 3 ml/well of DMEM/high-glucose complete medium was added thereto. The cells were proliferated in an incubator at 37° C. and 5% CO₂ for 24 hours until becoming approximately 80% confluent in observation under inverted microscope. The pseudotyped recombinant lentiviral particle THTN, THTH or THTC prepared as described in Example 2, or viral particle NL4-3 or D64V prepared by the transfection of human embryonic kidney 293T cells with pNL4-3 or pD64V was added to the wells (two wells for each lentiviral particle) in which the HeLa/tat cells were proliferated, to infect the cells by each virus. A virus solution having approximately 4×10⁵ infection units equivalent to 400 ng of p24 viral protein was used per 2×10⁵ target cells in each infection (multiplicity of infection (m.o.i.): 1.3).

On the other hand, Mock infection was performed by the incubation of cells with “dead” THTN virions. The THTN virions were prepared by boiling at 100° C. for 5 minutes to make sure that the virus is “dead.” The Mock infection with the “dead” virus guarantees that there is absolutely no viral particle entry into the cell, so there is no viral RNA transcribed in the RT-PCR assay.

The cells thus infected were proliferated in 5% CO₂ incubator at 37° C. for 8 hours. The cells were observed under microscope to confirm that the cells were healthy and were not contaminated. The medium was carefully discarded and 3 ml/well of fresh DMEM/high-glucose complete medium was added to the cells. The cells were proliferated in an incubator at 37° C. and 5% CO₂ for 48 hours. Then, the cells were rinsed three times using 1×PBS and scraped off from the surface of the wells using Rubber Policeman to collect the cells. The cells were collected into 15-ml low-speed centrifuge tube, to which 1×PBS was then added up to 12 ml in total. The tube was centrifuged at 3,000 rpm to form cell pellet. The supernatant was discarded and the cell pellet was frozen in dry ice for 30 minutes.

### 2. RNA Extraction

GTC buffer was prepared as follows: 212 g of guanidine thiocyanate (mw. 118.16), 2.2 g of sodium citrate (mw. 294.1) and 15 ml of sarkosyl (10% w/v) were dissolved in an appropriate amount of ddH₂O and ddH₂O was added to the solution up to 300 ml in total (final concentrations: 6 M, 25 mM and 0.5%, respectively). 1.4 ml of β-mercaptoethanol was added per 20 ml before use to prepare GTC buffer.

1,000 μl of the GTC buffer was added to the cell pellet in each tube. The cells were passed through 18 G 1/2 needle five times using a syringe to homogenize the cells.

Then, 1,000 μl of water-saturated phenol and 1,000 μl of chloroform were added to each tube and the tube was vortexed. Then, 3 ml of 1 M sodium acetate, pH 5.3 was added to each tube and the tube was vortexed and centrifuged at 9,000×g and 4° C. for 10 minutes. The formed upper layer solution was transferred to fresh 50-ml tube, to which 0.6 to 1.0 volume of isopropanol was then added. The solution was mixed by inverting the tube five times and left overnight at room temperature. The tube was vortexed for 30 seconds. 200 to 300 μl of the solution was transferred to 1.5-ml microtube and centrifuged at 13,000 rpm and 4° C. for 15 minutes to form RNA pellet. The supernatant was discarded and the RNA pellet was rinsed with 1 ml of 75% ethanol and centrifuged at 13,000 rpm and 4° C. for 5 minutes. The supernatant was discarded and the RNA pellet was dried in air.

### 3. RT-PCR

The RNA pellet was redissolved in 10 μl of ddH₂O. Reverse transcription reaction and PCR (RT-PCR) were performed for cDNA amplification. In this RT-PCR, 200 μg of RNA was used for each reverse transcription.

The reverse transcription (RT) reaction was performed as follows: 10 μl of DNase I-digested RNA (approximately 200μg), 1 μl of 10 mM dATP/dCTP/dGTP/dTTP stock, 1 μl of RNasein® (30 units/μl, Promega), 2 μl of 10×PCR buffer, 1 μl of 0.1 μg/μl random primer (random hexamer) and 1 μl of 50 mM MgCl₂ were gently mixed and then 1 μl of Mo-MuLV reverse transcriptase (200 units/pi) was added thereto and mixed in the tube by centrifugation for 2 seconds. The tube was incubated first at room temperature for 10 minutes and then at 42° C. for 60 minutes. Then, the tube was boiled in a water bath of 100° C. for 15 minutes and cooled on ice for 5 minutes. The RT reaction solution in the tube was aliquoted to four fresh tubes (in an amount corresponding to 50 μg of RNA for each tube) and stored at −80° C. for further use.

Next, the PCR reaction was performed as follows: 2.5 μl of primer 5′ LTRU5 (20 μm), 2.5 μl of primer 3′ NL5850 (20 μm) or primer 3′ NL8960, 5 μl of RT reaction solution and 40 μl of PCR mixture were mixed and subjected to the following temperature conditions using a thermal cycler for PCR reaction (Applied Biosystems): 1 cycle involving 94° C. for 4 minutes, and 16 cycles each involving 94° C. for 60 seconds, 60° C. for 30 seconds and 72° C. for 90 seconds, followed by 72° C. for 10 minutes.

The nucleotide sequences of the primers used are shown below.

Primer 5′ LTRU5:
(SEQ ID NO: 16)
5′-TCTGGCTAACTAGGGAACCCACTG-3′
Primer 3′ NL5850:
(SEQ ID NO: 17)
5′-GCTATGTCGACACCCAATTCTGAA-3′
Primer 3′ NL8960:
(SEQ ID NO: 18)
5′-TGTGCTTCTAGCCAGGCACAAGC-3′

The PCR mixture (for six samples) was prepared at a final volume of 240 μl by mixing the followings:

| | | |
|---|---|---|
| 10 mM dATP/dCTP/dGTP/dTTP stock | 6 | μl |
| ³²P-α-dCTP (3000 Ci/mmol, 10 μCi/μl, GE, USA) | 1 | μl |
| 10 × PCR buffer | 30 | μl |
| Taq DNA polymerase (5 units/μl) | 3 | μl |
| ddH₂O | 201 | μl |

After PCR, 3 μl of DNA loading buffer was added to 20 μl of each PCR reaction product. The mixture was loaded onto 2.2% agarose gel and electrophoresed in 1×TAE. The gel was wrapped, dried, and exposed to an X-ray film overnight at −80° C. The X-ray film was developed and photographs were taken. The 1×TAE was prepared by: dissolving 242 g of Tris Base (molecular biological grade), 57.1 ml of glacial acetic acid (molecular biological grade) and 100 ml of 0.5 M EDTA, pH 8.0 (molecular biological grade) in an appropriate amount of ddH₂O; adding ddH₂O to the solution up to 1,000 ml in total; and diluting 50×TAE thus prepared 50-fold with ddH₂O.

In the HeLa/tat cells infected by different recombinant lentiviral particles, splicing of lentiviral RNAs derived from these particles was compared. The viral particle D64V deficient in HIV-1 integrase and thus deficient in HIV-1 proviral DNA integration was used as a negative control.

### 4. Results

The results of PCR using the primers 5′ LTRU5 and 3′ NL5850 are shown in FIG. 8B. HIV-1 has seven major spliced RNA fragments bound to form various viral mRNAs. As a result of using the primers 5′ LTRU5 and 3′ NL5850, major PCR products of five out of these fragments were detected in all the tested cells infected by the lentiviral particles. Particularly, the cells infected by the viral particle THTN comprising the hTERT promoter in the viral genomic RNA exhibited very high RNA splicing activity, which was at least 100 times higher than the RNA splicing level of the cells infected by the wild-type NL4-3. By contrast, the RNA splicing level was not increased in the cells infected by the recombinant lentiviral particle THTH (lacking the 5′ portion (1 kb) of the hTERT promoter) or THTC (having the CMV promoter), compared with the cells infected by the wild-type viral particle NL4-3.

The results of PCR using the primers 5′ LTRU5 and 3′ NL8960 are shown in FIG. 8A. The PCR amplification level obtained using the primers 5′ LTRU5 and 3′ NL8960 represents the amount of viral genomic RNA derived from each lentiviral particle. However, PCR using the primers 5′ LTRU5 and 3′ NL8960 failed to efficiently produce PCR products and exhibited very weak signals. The cells infected by the viral particle THTN comprising the hTERT promoter in the viral genomic RNA had a viral genomic RNA level equivalent to that of the cells infected by other viral particles (THTH, THTC or NL4-3).

The results described above demonstrated that lentiviral RNA splicing is enhanced in the cells infected by the pseudotyped recombinant lentiviral particle carrying the lentiviral RNA comprising the human telomerase reverse transcriptase (hTERT) promoter, thereby enhancing transgene expression.

### Example 6 Enhancement of Gene Transduction and Expression by hTERT Promoter

The effect of hTERT promoter on gene transduction activity was examined by p24 ELISA assay using viral protein expression as an indicator.

293T cells and HeLa/tat cells inoculated at a concentration of 2×10⁶ cells were infected (multiplicity of infection (m.o.i.): 1.0) by the pseudotyped recombinant lentiviral particle THTN, THTH or THTC prepared in Example 2, or the viral particle NL4-3 or D64V. Upon infection, virus genomic DNA is synthesized from viral genomic RNA in the lentiviral particle and integrated as proviral genomic DNA into host cell genome. Proteins, for example, p24 viral antigen, expressed as a result of the gene integration (gene transduction) into host genome can be secreted into a culture medium. Thus, the medium was collected and the amount of the virus-derived protein p24 in the medium was measured using HIV-1 p24 antigen ELISA assay kit (Coulter Inc., Miami, Fla., USA) to examine the gene transduction activity of each viral particle.

First, the cell culture medium was collected at different points of times after the infection. Each medium collected was centrifuged to remove cell debris and then was diluted with 1×PBS buffer and subjected to p24 assay. The dilution ratio was usually set to 1:50 to 1:200. As a result, the p24 level was elevated in approximately 36 hours after the infection, reached a peak at 72 hours after the infection, and rapidly declined at 96 hours after the infection. Thus, the p24 level was indicated by an average in the medium collected 72 hours after the infection.

The results are shown in FIG. 9. The 293T cells infected by the viral particle THTC comprising the CMV promoter in the viral genomic RNA exhibited a p24 level higher than that of the 293T cells infected by the wild-type NL4-3. This indicates that the CMV promoter activates gene transduction and expression in the 293T cells. Such activation of gene transduction and expression was more apparent as to the viral particle THTN, which comprised the hTERT promoter in the viral genomic RNA and exhibited active RNA splicing as shown in Example 5. The 293T cells infected by THTN had a p24 level exceeding that of the 293T cells infected by THTC. By contrast, the 293T cells infected by the viral particle THTH lacking the 5′ portion (1 kb) of the hTERT promoter sequence had a p24 level equivalent to that of the cells infected by the wild-type NL4-3. Moreover, p24 was hardly detected as to D64V, which does not cause gene transduction, demonstrating that the p24 level reflects gene transduction and expression levels.

Next referring to the HeLa/tat cells, the cells infected by THTC or THTH produced p24 at a level equivalent to that in the case of the wild-type NL4-3. Only the HeLa/tat cells infected by the viral particle THTN comprising the hTERT promoter in the viral genomic RNA had a p24 level exceeding 2,500 ng/ml, which was obviously higher than that in the case of other viral particles.

The results described above demonstrated that gene transduction and expression are enhanced by the hTERT promoter, as is evident from the fact that viral protein expression was enhanced in the cells infected by the pseudotyped recombinant lentiviral particle carrying the lentiviral RNA comprising the hTERT promoter.

### Example 7 Enhancement of Microvesicle Release Based on hTERT Promoter

Cells into which the lentiviral plasmid vector pRBL0213T was introduced or cells infected by the viral particle RBL0213T were examined for their microvesicle (my) release using, as an indicator, the activity value of transgene PTEN in the cells or in mv determined by PTEN assay.

### 1. Plasmid DNA Transfection

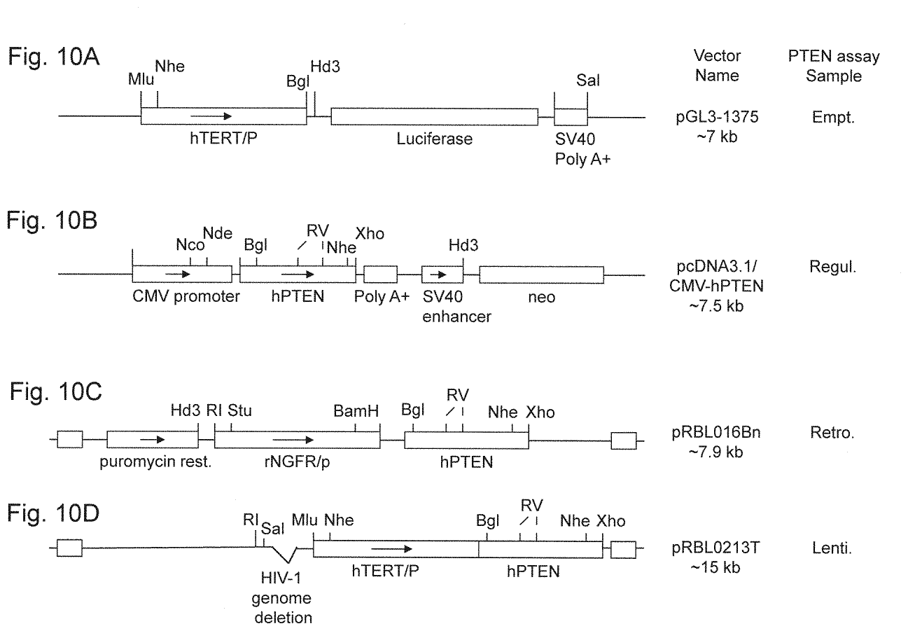

200 μl of 1 μg/ml pRBL0213T and 50 μl of 3 M sodium acetate, pH 5.3 were added to 1.5-ml microtube, to which 1 ml of 100% ethanol was then added. The solution was mixed and cooled at −80° C. for 60 minutes. Then, the tube was centrifuged at 13,000 rpm for 15 minutes to form DNA pellet. The supernatant was discarded. 1 ml of 70% ethanol was added to the tube, which was then centrifuged at 13,000 rpm for 5 minutes. The supernatant was carefully discarded and the DNA pellet was dried in air. The DNA pellet was dissolved by the addition of 500 μl of ddH₂O to prepare pRBL0213T plasmid DNA solution. The pRBL0213T is a recombinant lentiviral plasmid vector having the PTEN gene under control of the hTERT promoter (FIG. 10D, Lenti.).

Also, plasmid DNA solutions of plasmids pGL3-1375, pcDNA3.1/CMV-hPTEN and pRBL016Bn were prepared. The pGL3-1375 (Takakura et al., Cancer Res., 1999, p. 551-557) had hTERT promoter but no PTEN gene, and was used as a negative control of PTEN assay (FIG. 10A, Empt.). The pcDNA3.1/CMV-hPTEN was plasmid in which PTEN gene was inserted under control of CMV promoter of general plasmid pcDNA3.1, and was used as a positive control of PTEN assay (FIG. 10B, Regul.). The pRBL016Bn is a Mo-MLV retroviral plasmid having PTEN gene under control of a rat nerve growth factor receptor (rNGFR) gene promoter (FIG. 10C, Retro.).

500 μl of any of these four kinds of plasmid DNA solutions (20 μg of plasmid DNA), 400 μl of ddH₂O and 75 μl of 2 M CaCl₂ were added to fresh 50-ml tube and gently mixed. Then, 525 μl of 2×HEPBS was added dropwise thereto with gentle stirring to circumvent the formation of large precipitates. The tube was left at room temperature for 20 minutes to obtain a transfection mixture.

The medium was discarded from T75 flask in which human embryonic kidney 293T cells were proliferated as described in Example 2. Then, 12 ml of fresh DMEM/high-glucose complete medium was added to each flask. 1,000 μl of the transfection mixture was gradually added to each flask. The cells were incubated in 5% CO₂ incubator at 37° C. for 8 hours to transfect the cells with each plasmid DNA. Then, the medium was discarded and 15 ml of fresh DMEM/high-glucose complete medium was added to each flask. The cells were incubated in 5% CO₂ incubator at 37° C. for 60 hours. Also, mock transfection was performed by similar procedures using water instead of the plasmid DNA solutions.

### 2. Infection by Viral Particle RBL0213T

As described in Example 2, recombinant lentiviral particle RBL0213T was prepared using the plasmid pRBL0213T, then PEGylated, and purified. 293T cells, CEM cells or HeLa cells were infected (m.o.i.: 1.3) by the resulting recombinant lentiviral particle RBL0213T. The cells were infected by the viral particle RBL0213T by incubation for 12 hours. A fresh medium was added thereto and the cells were proliferated for 48 hours.

On the other hand, the chemically inactivated viral particle was prepared by: preparing 100 mM AT-2 (2,2′-dipyridyl disulfide) (aldrithiol-2) in DMSO (Fluka); adding the AT-2 at a concentration of 1 mM to the solution containing the viral particle; and treating the mixture overnight at 4° C. (Rossio J L, J. Virol., 1998, Vol. 72, p. 7992-8001). The AT-2 is a reagent that oxidizes cysteine in proteins in virions to inhibit the functions of reverse transcriptase, thereby inactivating the virus. The AT-2 treatment deletes the infectivity of HIV, though the integrity and conformation of viral surface proteins are maintained.

### 3. Preparation of Cell Lysate

A solution containing free phosphate is not preferable for PTEN assay because of producing high backgrounds. Buffers and tubes are recommended to be phosphate-free.

After the transfection or the infection described above, the cells were collected into fresh 50-ml tube and centrifuged at 3,000 rpm for 5 minutes to precipitate the cells. For use in “4. Preparation of mv lysate” described later, the medium was transferred to fresh 50-ml tube. The precipitated cells were rinsed with 30 ml of cold 1×PBS and centrifuged again to precipitate the cells. The supernatant was discarded. 250 μl of lysis buffer (25 mM Tris-HCl, pH 8.0, 150 mM NaCl, 1% NP-40, 1 mM EDTA, 5% glycerol) was added to the cells (2×10⁶ cells). The cells were passed through 24 G needle 10 times using a syringe to homogenize the cells, thereby obtaining cell extracts.

The cell extracts were incubated at 4° C. for 60 minutes on a rotary platform and centrifuged at 13,000 rpm and 4° C. for 20 minutes. The supernatant (cell lysates) was transferred to a fresh tube. 5 μl of the cell lysates was added to another tube. Then, 1 ml of Bio-Rad protein assay solution (Bio-Rad, USA) was added thereto and OD₆₀₀ was measured. Bovine serum albumin was used as a standard to determine a protein concentration. The remaining cell lysates were stored at −80° C. for further use.

### 4. Preparation of My Lysate

The culture medium after the transfection or the infection transferred to the 50-ml tube in “3. Preparation of cell lysate” described above was centrifuged at 9,000×g and 4° C. for 60 minutes. The supernatant was transferred to a fresh tube and ultrafiltered using Vivaspin® 20 (1,000 kDa mw. co.) (Sartorius, Bohemia, NY, USA), thereby replacing the buffer with TBS-D. The TBS-D was prepared by: mixing 6.5 ml of 1 M Hepes-KOH, pH 7.6, 7 ml of 5 M NaCl and 0.25 ml of 1 M KCl with an appropriate amount of ddH₂O; adding ddH₂O to the mixture up to 250 ml in total; and, to the TBS solution thus prepared, adding 500 μl of 1 M dithiothreitol (DTT) per 50 ml of the TBS solution before use.

To 1 to 2 ml of the solution thus prepared from the medium, the same volume thereas of PEG 8,000/NaCl solution was added and the mixture was incubated overnight at 4° C. on a rotary platform. The PEG 8,000/NaCl solution (300 ml) was prepared by: mixing 90 g of PEG 8,000 and 180 ml of 5 M NaCl with an appropriate amount of ddH₂O; and adding ddH₂O to the mixture up to 300 ml (final volume).

The solution thus incubated was centrifuged at 13,000 rpm for 20 minutes to form pellet containing microvesicles (my). The supernatant was discarded and the pellet was resuspended in 65 μl of lysis buffer and incubated at 4° C. for 2 hours on a rotary platform to lyse the pellet. This solution was centrifuged at 13,000 rpm for 20 minutes and the supernatant (my lysates) was transferred to a fresh tube. 5 μl of the my lysates was added to another tube and OD₆₀₀ was measured to determine a protein concentration. The remaining mv lysates were stored at −80° C. for further use.

### 5. PTEN Immunoprecipitation (IP)

70 μl of the cell lysates or the mv lysates (containing 500 μg of the protein) and 5 μl of mouse anti-PTEN monoclonal antibody (clone 6H2.1, 1 mg/ml, Upstate, USA) were added to a fresh tube, to which TBS-D was then added up to 100 μl (final volume) to prepare each IP sample. The IP sample was incubated overnight at 4° C. on a rotary platform. Then, 50 μl of prewashed protein A-agarose solution (Thermo Scientific, Illinois, USA) was added to each IP sample and the mixture was incubated overnight at 4° C. on a rotary platform. After the incubation, the agarose beads were washed with 1 ml of TBS-D and centrifuged at 6,500 rpm for 5 minutes. The washing was further repeated three times. After the centrifugation, the supernatant was discarded and the agarose beads were resuspended in 75 μl of TBS-D to prepare an agarose bead solution. The solution was stored at 4° C. for further use.

### 6. PTEN Assay

PTEN assay was conducted by a partial modification of Echelon PTEN phosphatase malachite green assay (Echelon Biosciences, Utah, USA). For each assay, the final volume was brought up to 100 μl from the original volume of 25 μl and absorbance at 620 nm was measured using Promega GloMax-Multi Jr. reading system (Promega, USA). The assay was conducted using liquid substrate PtdIns(3,4,5)P3 (Echelon, Utah, USA) according to the manual of the manufacturer. For each assay, 23 μl of TBS-D buffer, 70 μl of the agarose bead solution obtained in “5. PTEN immunoprecipitation (IP)” described above and 7 μl of 1 mM PtdIns(3,4,5)P3 stock solution were mixed. The mixture was incubated at 37° C. for 2 hours. 450 μl of malachite green solution (Echelon, Utah, USA) of room temperature was added to the mixture. The tube containing the mixture was covered with aluminum foil for protection from light and incubated at room temperature for 30 to 60 minutes. Absorbance at 620 nm was measured using a malachite green solution as a blank and a substrate as a phosphatase background. PTEN activity values were obtained from three different transfections or infections. The activity values of the substrate (background) were subtracted from the obtained values and an average of the resulting values was obtained.

### 7. Results

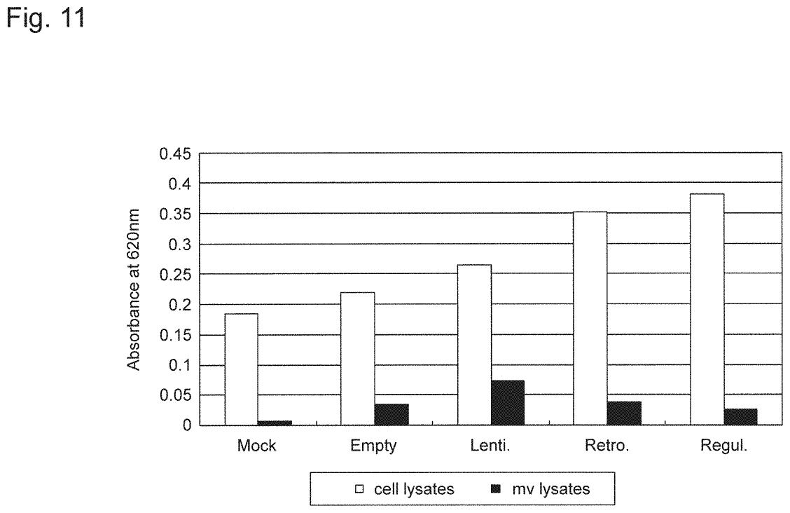

The results of PTEN assay on 293T cells transfected with each plasmid are shown in FIG. 11. The highest PTEN activity in the cell lysates was confirmed for the cells transfected with the Regul. vector comprising the strong CMV promoter that drove the transcription of the PTEN gene. The PTEN activity was also high in the case of the transfection with the Retro. vector comprising the rNGFR promoter that drove the transcription of the PTEN gene. The transfection with the Lenti. vector comprising the hTERT promoter that drove the transcription of the PTEN gene gave a moderate PTEN activity value, compared with the Regul. and Retro. vectors. As for the mv lysates, the highest PTEN activity was confirmed in the case of the transfection with the Lenti. vector, compared with the Regul. and Retro. vectors.

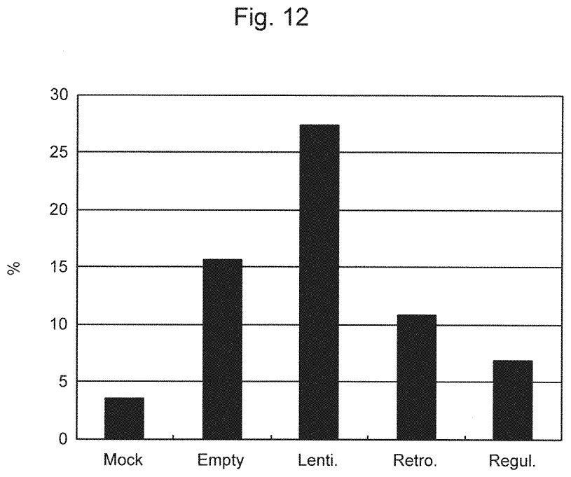

FIG. 12 shows the ratio of the PTEN activity in the mv lysates to the PTEN activity in the cell lysates shown in FIG. 11. The transfection with the Lenti. vector gave the highest ratio of the PTEN activity in the mv lysates (27.37%). Moreover, it is to be noted that the much higher ratio of the PTEN activity in the my lysates was shown from the transfection with the Empt. vector, which comprised the hTERT promoter as in the Lenti. vector for gene transcription and had no PTEN gene, compared with the Regul. and Retro. vectors. These results demonstrated that the transfection with the Empt. vector promotes the mv encapsulation of the endogenous PTEN of host cells whereas the transfection with the Lenti. vector promotes the my encapsulation of both endogenous and transgene products PTEN, thereby enhancing the release of mv into an extracellular environment.

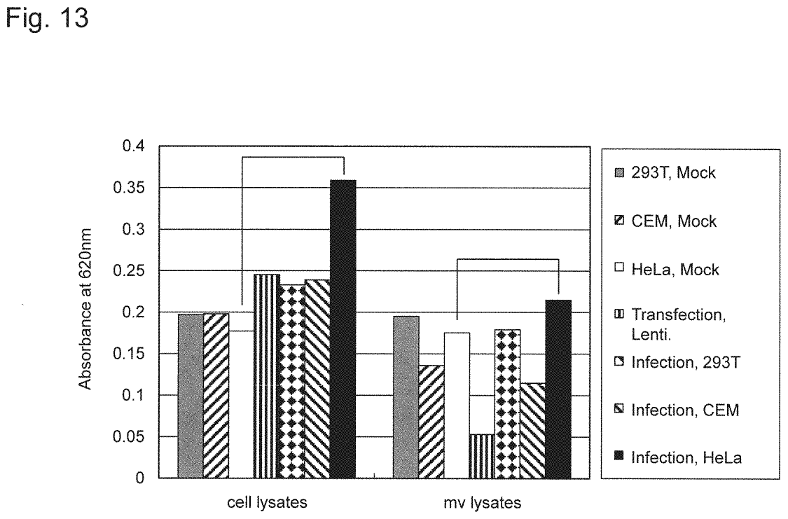

Further, the results of PTEN assay on 293T cells, HeLa cells and CEM cells infected by the viral particle RBL0213T are shown in FIG. 13. The cells incubated with the recombinant lentiviral particle RBL0213T released a larger number of mv than that in the case of transfection, irrespective of whether the particle was infectious or inactive (mock infection) (FIG. 13, mv lysates). By contrast, the cell lysates of the cells infected by the recombinant lentiviral particle RBL0213T or mock-infected had PTEN activity substantially equivalent to that in the case of transfection (FIG. 13, cell lysates). However, elevated PTEN activity was observed in the lysates of the HeLa cells infected by the viral particle RBL0213T. These results demonstrated that the infection by the recombinant lentiviral particle RBL0213T tends to increase PTEN activity in the mv lysates, unlike in the cell lysates, compared with the transfection with the Lenti. vector pRBL0213T. Further, the infection by the lentiviral particle was shown to cause the human uterine cervix cancer HeLa cells to form and release a larger number of transgene product PTEN-encapsulated my among the tested cells (FIG. 13, mv lysates). As a result of conducting PTEN assay on the solution of the viral particle RBL0213T, the PTEN activity value was very small (OD₆₂₀=0.065), demonstrating that PTEN is hardly incorporated in the viral particle.

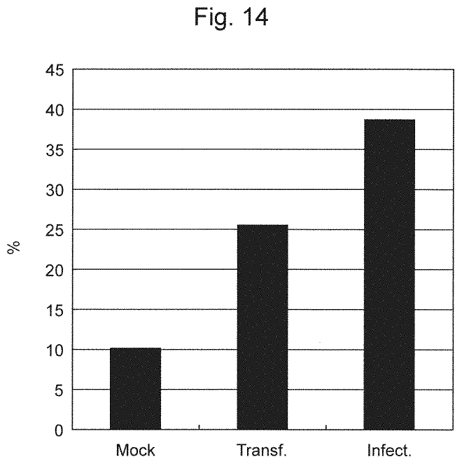

FIG. 14 shows the ratio of the PTEN activity in the mv lysates to the PTEN activity in the cell lysates of the 293T cells transfected with the recombinant lentiviral plasmid vector pRBL0213T or the 293T, CEM or HeLa cells infected by the recombinant lentiviral particle RBL0213T or mock-infected. The infection (Infect.) and the mock infection (Mock) are indicated by values that compensate for the nonspecific stimulation of mv release for the mock infection. The ratio of the PTEN activity in the mv lysates to the PTEN activity in the cell lysates was larger for the infection than for the mock infection, demonstrating that the infection by the recombinant lentiviral particle RBL0213T enhanced mv release from the cells. In contrast to the mv release ratio of 27.37% in the cells transfected with the Lenti. vector (FIG. 12), the mv release ratio reached 38.75% in the cells infected by the recombinant lentiviral particle RBL0213T (FIG. 14), demonstrating that the infection by the recombinant lentiviral particle RBL0213T further significantly promoted mv release.

The results described above demonstrated that the cells transfected with the recombinant lentiviral plasmid vector comprising the hTERT promoter in the lentiviral RNA intracellularly produce a large amount of microvesicles and exhibit the enhanced release of microvesicles carrying the transgene product. The cells infected by the lentiviral particle prepared using such a plasmid were shown to exhibit the more strongly enhanced microvesicle release.

### Example 8 Preparation of Microvesicle

The microvesicle (my) of the present invention was prepared by the method shown below.

### 1. Plasmid DNA Transfection or Viral Infection

15 ml of DMEM/high-glucose (Hyclone, Utah, USA) complete medium (supplemented with 10% fetal bovine serum, 100 units/ml penicillin and 100 μg/ml streptomycin (Hyclone, Utah, USA)) was added to T75 flask, to which human embryonic kidney 293T cells were then inoculated. The 293T cells were proliferated in 5% CO₂ incubator at 37° C. and increased to cultures in ten T75 flasks while subcultured. The 293T cells were subjected to plasmid DNA transfection or viral infection shown below.

### (A) Plasmid DNA Transfection

The followings were added to 50-ml tube:
(a) 500 μl of plasmid DNA solution containing 170 μg of the recombinant lentiviral plasmid vector pTHTN or pRBL001 described in Example 1,
(b) 1,650 μl of ddH₂O, and
(c) 350 μl of 2 M CaCl₂).

The solution was gently mixed and then 2,500 μl of 2×HEPBS was added dropwise thereto with gentle stirring to circumvent the formation of large precipitates, thereby preparing a transfection mixture. The tube containing this transfection mixture was left at room temperature for 20 minutes. The medium was discarded from the ten T75 flasks in which the 293T cells were proliferated. Then, 12 ml of fresh DMEM/high-glucose complete medium was added to each flask. 500 μl of the transfection mixture was gradually added to each flask. The cells were incubated in 5% CO₂ incubator at 37° C. for 8 hours.

### (B) Viral Infection

The 293T cells were infected by coincubation in 5% CO₂ incubator at 37° C. for 16 hours with the pseudotyped recombinant lentiviral particle THTN or THTD prepared as described in Example 2 (multiplicity of infection: 0.3-0.4).

### 2. Cell Culture and Collection of Medium

The medium was discarded and 15 ml of fresh DMEM/high-glucose complete medium was added to each flask. The cells were incubated in 5% CO₂ incubator at 37° C. for 60 hours and the medium was collected.

### 3. Purification

50-ml tube containing the medium collected was centrifuged at 3,000 rpm and 4° C. for 5 minutes. The medium supernatant thus centrifuged was transferred to 250-ml high-speed centrifuge tube and centrifuged at 9,000×g and 4° C. for 60 minutes. The resulting supernatant was concentrated 5-fold by ultrafiltration using Vivaspin® 20 (1,000 kDa molecular weight cutoff (mw. co.)) (Sartorius, NY, USA). MMP solution was added to the resulting solution and mv was precipitated overnight at 4° C.

The solution in which mv was precipitated was centrifuged at 9,000×g and 4° C. for 30 minutes to form pellet containing my. The supernatant was discarded and the pellet was resuspended in 10 ml of 1×PBS to obtain a solution containing mv.

mv in an aliquot of the solution was PEGylated (Croyle et al., J. Virol., 2004, Vol. 78, p. 912-921). The PEGylation was performed by adding 0.4 ml of PEGylation solution (33 mg/mL methoxy PEG succinimidyl carbonate NHS (mPEG-NHS, m.w. 10K (NANOCS, USA)), 30 mM HEPES-KOH, pH 7.5, 500 mM NaCl) to approximately 10 ml of the solution containing mv and incubating the mixture at room temperature for 60 minutes on a rotary platform.

The solution containing mv PEGylated or unPEGylated was dialyzed against 1×PBS at 4° C. using Slide-A-Lyzer cassette (20 kDa mw. co.) (Thermo Scientific, IL, USA) and 1×PBS was replaced with fresh one every 24 hours for 2 days. The mv solution thus dialyzed was concentrated 30-fold using AmiconUltra 15 (100 kDa mw. co.) (Millipore, MA, USA). This mv solution concentrated was passed through a syringe filter with 0.45 μm pore size and the resulting preparation was stored at −80° C.

In this way, microvesicle Lenti-mv2010 carrying the partial fragment of SV40 large T antigen as a transgene product was prepared using the recombinant lentiviral plasmid vector pTHTN or the pseudotyped recombinant lentiviral particle THTN. Further, microvesicle Lenti-mv2010/CDC6 shRNA carrying the transgene product CDC6 shRNA was prepared using the recombinant lentiviral plasmid vector pRBL001 or the pseudotyped recombinant lentiviral particle THTD.

### Example 9 Detection of Protein Contained in Microvesicle

In this Example, proteins contained in microvesicles were examined. Also, the microvesicle-mediated delivery of substances into cells was examined.

Human embryonic kidney 293T cell were transfected with plasmids (pcDNA3.1 backbone) expressing N-terminally c-myc-tagged human DNA flap endonuclease 1 (c-myc-FEN-1) to ectopically express c-myc-tagged FEN-1. The FEN-1 has been shown to function as an important cellular helper factor for the maturation of HIV-1 virus genomic DNA and its integration into host genome. The microvesicle Lenti-mv2010 derived from the viral particle THTN-infected cells prepared as described in Example 8 was coincubated with some of the 293T cells transfected with the c-myc-FEN-1 expression plasmid or with untransfected 293T cells. After incubation for 60 hours, nuclear and cytoplasmic extracts were prepared from the cells. Further, the cell culture medium was collected and mv was prepared by the method (except for PEGylation) described in “3. Purification” of Example 8. The resulting nuclear and cytoplasmic extracts and mv were analyzed by Western blot.

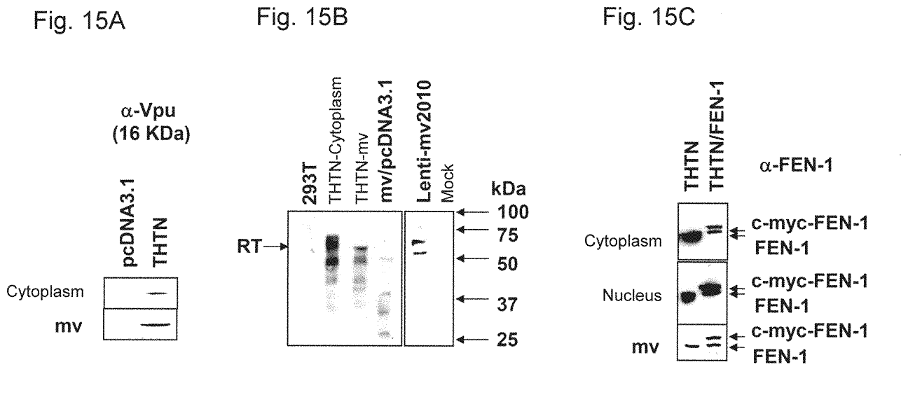

The results of detecting Vpu protein using anti-HIV-1 Vpu antibody are shown in FIG. 15A. The Vpu protein was detected in the cytoplasmic extracts of the 293T cells that were transfected with the c-myc-FEN-1 expression plasmid and then coincubated with Lenti-mv2010, and in mv released from the cells (FIG. 15A, THTN), whereas this protein was not detected in the 293T cells that were transfected with c-myc-FEN-1 expression plasmid but were not coincubated with Lenti-mv2010 (FIG. 15A, pcDNA3.1). The Vpu is a protein that suppresses the tetherin- or CD317-mediated attachment of virions to cell membranes, thereby enhancing the release of the HIV-1 virions (Sauter et al., Cell, 2010, Vol. 141, p. 392-398). This showed that: the microvesicle Lenti-mv2010 derived from the 293T cells infected by the recombinant lentiviral particle THTN comprises the viral protein Vpu; the contents were delivered to other cells via the microvesicle; and microvesicles released from the cells that received the delivery also comprised the Vpu protein.

The results of detecting RT protein using anti-HIV-1 reverse transcriptase (RT) antibody are shown in FIG. 15B. The RT protein was detected in the microvesicle Lenti-mv2010 derived from the 293T cells infected by the recombinant lentiviral particle THTN (FIG. 15B, Lenti-mv2010). By contrast, the RT protein was not detected in mv derived from the 293T cells mock-infected by the viral particle THTN chemically inactivated by AT-2 (FIG. 15B, Mock). The RT protein was also detected in the cytoplasmic extracts (FIG. 15B, THTN-cytoplasm) of the 293T cells that were transfected with the c-myc-FEN-1 expression plasmid and then incubated with Lenti-mv2010, and in mv (FIG. 15B, THTN-mv) prepared from the cells. By contrast, the RT protein was detected neither in mv (FIG. 15B, my/pcDNA3.1) prepared from the 293T cells that were transfected with the c-myc-FEN-1 expression plasmid but were not coincubated with Lenti-mv2010 nor in the untreated 293T cells (FIG. 15B, 293T). This showed that: the microvesicle Lenti-mv2010 derived from the 293T cells infected by the recombinant lentiviral particle THTN comprises the viral protein RT; the contents were delivered to other cells via the microvesicle; and microvesicles released from the cells that received the delivery also comprised the RT protein.

The results of detecting endogenous FEN-1 and foreign FEN-1 using anti-FEN-1 antibody are shown in FIG. 15C. The endogenous FEN-1 and the foreign FEN-1 (i.e., c-myc-FEN-1) were detected in the nuclei and cytoplasms prepared from the 293T cells that were transfected with the c-myc-FEN-1 expression plasmid and then coincubated with Lenti-mv2010, and in mv released from the cells (FIG. 15C, THTN/FEN-1). As for the 293T cells that were coincubated with Lenti-mv2010 but were not transfected with the c-myc-FEN-1 expression plasmid, only the endogenous FEN-1 was detected in the nuclei and cytoplasms and in mv released from the cells (FIG. 15C, THTN). This showed that both the cell-endogenous protein (FEN-1) and the foreign protein (c-myc-FEN-1) are encapsulated in the microvesicle.

The results described above demonstrated that viral proteins and other cell-endogenous and foreign proteins are encapsulated in microvesicles, particularly, exosomes, probably with the aid of the viral protein Vpu and a cell-protein transport system called endosomal sorting complexes required for transport (ESCRT), in cells. These results also demonstrated that the contents encapsulated in microvesicles are delivered to other cells.

### Example 10 Microvesicle-Mediated Delivery of Substance

The delivery (gene transduction) of substances via purified PEGylated my was examined using cultured cells.

c-myc-FEN-1-encapsulated PEGylated microvesicle Lenti-mv2010/c-myc-FEN-1 was prepared according to the method for preparing microvesicles by THTN infection described in Example 8 except that the cells used were changed to 293T cells transfected with the c-myc-FEN-1 expression plasmid. Lenti-mv2010/c-myc-FEN-1 was introduced into human uterine cervix cancer HeLa cells by coincubation. The HeLa cells coincubated therewith were subjected to indirect immunofluorescence staining using anti-c-myc antibody.

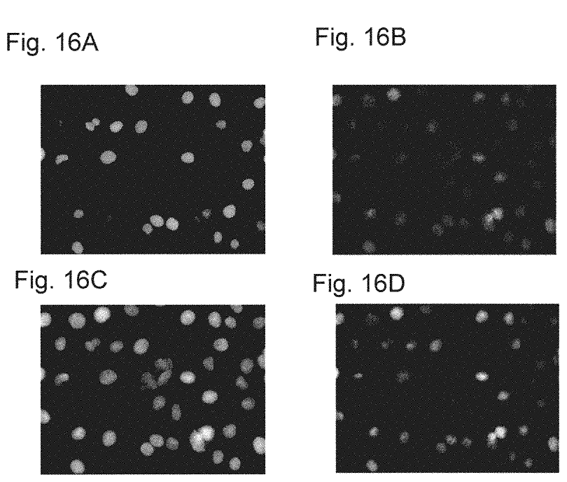

The results are shown in FIG. 16. FIG. 16A shows an anti-c-myc antibody stained image. FIG. 16B shows a DAPI stained image. FIG. 16C shows an overlaid image of FIGS. 16A and 16B. FIG. 16D shows an image prepared from the overlaid image by the color curve program in the image “adjustment” method of Photoshop® (Adobe Systems Inc.). In FIG. 16D, thicker red color represents increase in c-myc-FEN-1 level and greenish yellow color represents the absence of c-myc-FEN-1. Many red cells expressing c-myc-FEN-1 was seen in FIG. 16D, demonstrating that c-myc-FEN1 was delivered to a larger number of cells as a result of the coincubation with Lenti-mv2010/c-myc-FEN-1. The PEGylated microvesicles were shown to be taken up by many cells and be able to deliver the contents encapsulated in the microvesicles to other cells, because of being very stable and active.

### Example 11 Suppression of Cancer Cell Proliferation by Microvesicle Carrying CDC6 shRNA

Microvesicles carrying CDC6 shRNA (Lenti-mv2010/CDC6 shRNA) were tested for their cancer cell proliferation suppressive effect.

Human breast cancer MCF7 cells, human neuroblastoma LA-N-2 cells and neuroblastoma KANR cells were separately incubated with the microvesicle Lenti-mv2010/CDC6 shRNA carrying CDC6 shRNA prepared as described in Example 8. As a control, the microvesicle Lenti-mv2010 carrying the partial fragment of the SV40 large T antigen prepared as described in Example 8 was used. Then, proliferation of the cells was examined by MTT Cell Viability and Proliferation Assay Kit (ScienCell, USA). In this assay, the proliferation of cultured cells was quantified using, as an indicator, the absorbance of a substrate reduced in live cells.

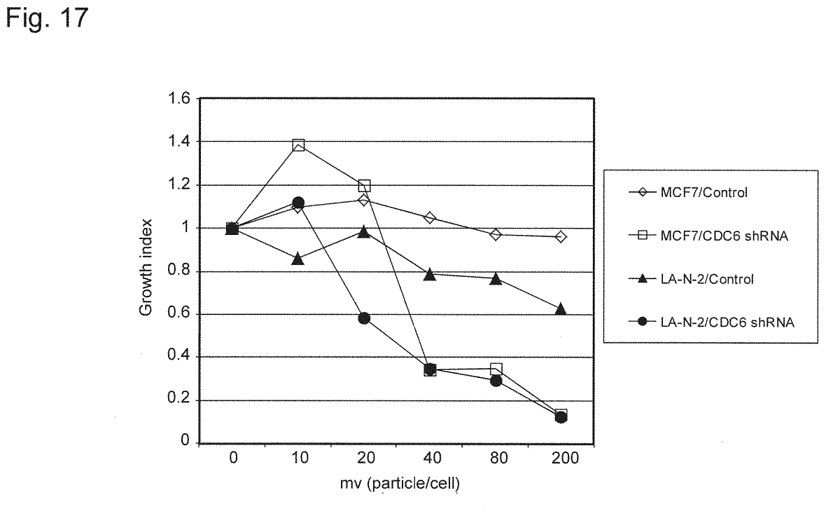

The results are shown in FIG. 17. The horizontal axis denotes the number of mv incubated per cell and the vertical axis denotes the ratio (growth index) of the absorbance value obtained to an absorbance value obtained in the absence of my incubation. The proliferation of the MCF7 cells and the LA-N-2 cells was significantly inhibited by the incubation with Lenti-mv2010/CDC6 shRNA, but was not inhibited by the incubation with Lenti-mv2010 (control) (FIG. 17). On the other hand, the proliferation of the KANR cells was not inhibited even by the incubation with Lenti-mv2010/CDC6 shRNA. The MCF7 cells and the LA-N-2 cells exhibited an elevated CDC6 expression, whereas CDC6 expression was hardly detected in the KANR cells. This showed that the CDC6 shRNA contained in the microvesicle knocked down CDC6, resulting in the inhibited proliferation of the cancer cells.

The results described above demonstrated that the microvesicle carrying CDC6 shRNA can deliver the CDC6 shRNA to CDC6-expressing cancer cells, thereby suppressing their proliferation.

### Example 12 Molecular Mechanism of Suppression of Cancer Cell Proliferation by Microvesicle Carrying CDC6 shRNA

The molecular mechanism underlying the suppression of cancer cell proliferation by Lenti-mv2010/CDC6 shRNA was examined. The previous study has revealed that: a high level of CDC6 protein is related to oncogenic activity in human cancer; and the protein levels of CDC6 and tumor suppressor p16^{INK4a} show an inverse correlation therebetween (Gonzalez, S. et al., Nature, 2006, p. 702-706). The progression of cell cycle of MCF7 cells is not inhibited even in the presence of p16^{INK4a}. This indicates the inactivation of the p16^{INK4a}-Rb pathway in the cancer cells. However, the p16^{INK4a}-Rb pathway may be reactivated in MCF7 cancer cells through Lenti-mv2010/CDC6 shRNA-mediated CDC6 knockdown. Thus, nonradioactive immunoprecipitation kinase assay was conducted to examine the reactivation of the p16^{INK4a}-Rb pathway. CDC6 is found in CDK4 kinase complex and required for Rb-C phosphorylation.

CDC6 in the MCF7 cells was removed (knocked down) by incubation with Lenti-mv2010/CDC6 shRNA. As a result, Rb-C phosphorylation was inhibited. The CDC6 knockdown increased the CDK inhibitory activity of p16^{INK4a} by at least 25 times. This showed the reactivation of the p16^{INK4a}-Rb pathway.

The results described above demonstrated that the microvesicle carrying CDC6 shRNA knocks down CDC6 in cancer cells and reactivates the p16^{INK4a}-Rb pathway, thereby functioning to suppress cancer cell proliferation.

### Example 13 Western Blotting Analysis

In this Example, PTEN protein was detected by Western blotting analysis in protein extracts of cells transfected with PTEN-encoding vectors and extracts of microvesicles obtained by culturing the cells. Plasmid vectors pGL3-1375, pcDNA3.1/CMV-hPTEN, pRBL016Bn and pRBL0213T as described in Example 7 and FIG. 10 and lentivirus particle vector RBL0213T as described in Examples 2 and 7 were used as a vector.

### 1. Preparation of Protein Extracts

About 1×10⁶ 293 T cells were transfected with above-mentioned different plasmid vectors. After 60 hours of transfection, cells were harvested, pelleted, homogenized and a supernatant was collected therefrom to prepare cellular protein extracts as described in the section “3. Preparation of cell lysate” of Example 7.

Further, the media after 60 hours of transfection were collected, and microvesicle lysates were prepared therefrom as described in the section “4. Preparation of mv lysate” of Example 7.

In addition, about 1×10⁶ 293 T cells were infected with lentiviral particle vector RBL0213T with m.o.i. at 0.3 in 25 ml of complete DMEM medium, as described in the section “2. Infection by viral particle RBL0213T” of Example 7. After infection for 60 hours, cellular protein extracts were prepared as described in the section “3. Preparation of cell lysate” of Example 7. Further, the media after infection for 60 hours were collected, and microvesicle lysates were prepared therefrom as described in the section “4. Preparation of mv lysate” of Example 7.

The prepared cellular protein extracts (about 20 micrograms of protein) or microvesicle lysates (about 50 micrograms of protein) were mixed with 2×SDS loading buffer (4% SDS, 250 mM Tris-HCl, pH 6.8, 3% β-mercaptoethanol, 15% glycerol, 0.05% bromophenol blue), and incubated in a boiling water bath for 15 minutes. Then, the protein samples are separated by electrophoresis in a 12% SDS-PAGE gel (Precise™ Protein Gel) using Thermo Scientific Owl Model P82 Minigel Protein Electrophoresis System. After the electrophoresis, the proteins were transferred from the gel to PVDF membrane, and probed with mouse anti-PTEN monoclonal antibody (primary antibody; Clone 6H2.1, MilliPore), followed by goat anti-mouse secondary antibody (1:5,000 dilution, MilliPore) to detect PTEN protein.

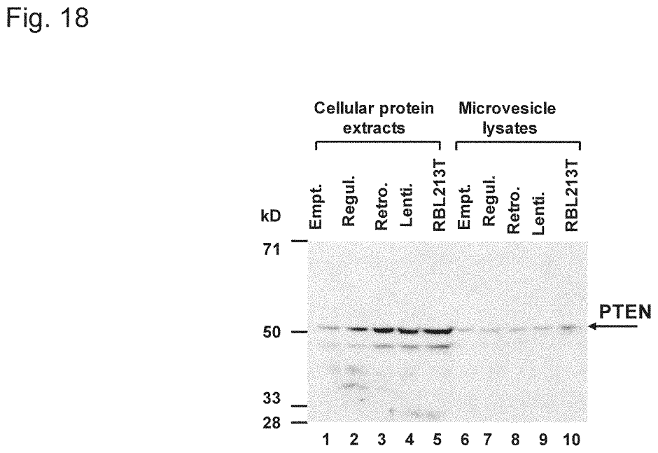

The detection results in the cellular protein extracts (lanes 1 to 5) and the microvesicle lysates (lanes 6 to 10) are shown in FIG. 18. Lanes 1 and 6 indicate cells transfected with pGL3-1375 (Empt.). Lanes 2 and 7 indicate cells transfected with pcDNA3.1/CMV-hPTEN (Regul.). Lanes 3 and 8 indicate cells transfected with pRBL016Bn (Retro.). Lanes 4 and 9 indicate cells transfected with pRBL0213T (Lenti.). Lanes 5 and 10 indicate cells infected with RBL0213T.

Endogenous PTEN was detected (lanes 1 and 6), and transgene product PTEN was detected in the cellular protein extracts (lanes 2 to 5) and microvesicle lysates (lanes 7 to 10). This result indicates that transgene product PTEN was produced, and encapsulated into microvesicles, and the microvesicles (genetically engineered microvesicles) were released from the cells.

The levels of the transgene product PTEN prepared from cells transfected with Regul., Retro., or Lenti (lanes 2 to 4) were increased by 2 to 3 times compared to that prepared from cells transfected with Empt. (lane 1). Further, the level of the transgene product PTEN prepared from cells infected with RBL0213T lentivirus vector (lane 5) was well above that of endogenous PTEN (lane 1).

In addition, the level of the transgene product PTEN in microvesicle lysates prepared using cells infected with RBL0213T lentivirus vector (lane 10) was clearly increased compared to those of the transgene product PTEN in the microvesicle lysates prepared using cells transfected with Empt., Regul., Retro., or Lenti (lanes 7 to 9). This indicates that the infection with virus particle-like lentivirus vector according to the present invention is more suitable for enhancing production and release of microvesicles that carry transgene products.

### Example 14 Inhibitory Effect of Genetically Engineered Microvesicles on Tumor Growth

In this Example, the effect of genetically-engineered microvesicles carrying a tumor-suppressor gene product (herein, also referred to as Cytomox) to inhibit the growth of tumor was examined by directly injecting the microvesicles into tumors.

### 1. Preparation of Test Sample

The following test samples were used for intra-tumor injection.

i) Cytomox HD (emvp 130001)

Based on the section “4. Preparation of mv lysate” of Example 7 and Example 8, human embryonic kidney 293T cells were transfected with both of the lentiviral vectors pRBL001 and pRBL0203, and the medium was collected by precipitating cultured cells, and microvesicles were prepared. The microvesicles were called Cytomox HD. Lentivirus vector pRBL001 is described in Example 1, and was used herein as a vector for producing CDC6 shRNA. Lentivirus vector pRBL0203 is a plasmid comprising human p16^{INK4a} gene which has been inserted under control of hTERT promoter in the pTHTK backbone and was produced in a similar way to Example 1. Specifically, pCMV p16INK4a (Plasmid 10916; SEQ ID NO: 25; Medema et al., Proc. Natl. Acad. Sci. USA., (1995) 92(14):6289-6293), which is the plasmid construct carrying p16^{INK4a} cDNA, was cleaved with EcoR I and Xho I, and the resulting fragment (p16 cDNA of about 0.45 kb) was subcloned in the Hind III-Xho I site of pEND-HTP1, in which the 1.5 kb hTERT promoter from pGL3-1375 has been cloned at Mlu I-BamH I site. The DNA fragment containing the hTERT promotor and p16INK4a cDNA downstream thereof was cleaved with Mlu I and Xho I, and the 2.0 kb fragment was inserted into the pTHTK backbone to produce the vector pRBL0203.

ii) Cytomox p53 (emvp 130003)

Based on Example 8, human embryonic kidney 293T cells were transfected with the vector pGLQ-p53EX, and the medium was collected by precipitating cultured cells, and microvesicles were prepared. The vector pGLQ-p53EX is a plasmid expression vector comprising human p53 gene (SEQ ID NO: 26; CDS of GenBank accession no. BC003596). Specifically, subclone vector pBS-TP53PU carrying the human p53 tumor suppressor cDNA of about 2.0 kb that is from the plasmid pBSH19 carrying p53 cDNA was linearized with Hind III at its 3′ end. The generated Hind III site was blunted, followed by Sal I digestion of the vector. The backbone was purified and ligated with an about 0.3 kb DNA fragment (with blunt-ended Xba I plus Sal I ends) isolated from pGL3-378 to form pBS-TP53PA. The p53 cDNA fragment was cleaved from pBS-TB53PA with Stu I to Sal I and inserted into pGL3-378 vector at blunt-ended Xba I-Sal I site to form pGLQ-3UTR. p53 cDNA of about 1.4 kb (containing entire coding region of human p53 gene) was cleaved from another subclone vector pUTK-p53TT with Hind III and Xho I, and inserted into pGLQ-3UTR to produce pGLQ-p53EX.

iii) Cytomox PTEN (emvp 130006)

Based on the section “4. Preparation of mv lysate” of Example 7 and Example 8, human embryonic kidney 293T cells were transfected with the lentiviral vector pRBL0213T, and the medium was collected by precipitating cultured cells, and microvesicles were prepared. The microvesicles were called Cytomox PTEN. Lentivirus vector pRBL0213T is described in Example 1, and was used herein as a vector for producing human PTEN protein.

### iv) Cytomox EX (T 130075)

As described in Example 2, recombinant lentivirus particles RBL001 and RBL0203 were produced by using plasmids pRBL001 and pRBL0203. Based on the section “4. Preparation of mv lysate” of Example 7 and Example 8, human embryonic kidney 293T cells were infected with the lentivirus particle, and the medium was collected by precipitating cultured cells, and microvesicles were prepared. Specifically, the cells were transfected with both of the lentivirus vectors RBL001 and RBL0203 as described in Example 8, and after 60 hours of infection, the medium was collected and the cell debits were removed by centrifugation. The medium was subjected to high speed centrifugation at 9,000 g, 4° C. for 60 minutes. The supernatants were collected for purifying microvesicles as described in Example 8, and the trace amount of pellets were resuspended in 1×PBS and subjected to dialysis followed by concentration with Amicon Ultra 15 (100 kDa mw. co.) (Millipore). The resulting preparation (microvesicles) was called Cytomox EX.

### v) Recombinant Interferon α-2b (Positive Control)

Recombinant interferon α-2b (Schering-Plough (Brinny) Co., Ireland) was used as positive control.

### 2. Animals

BALB/c-nu mice (female, total eighty animals, grade SPF, 14-16 grams each) were bought from Laboratory Animal Center of Chinese Academy of Medical Sciences for using in the Example.

All of mice were kept for breeding in animal room with an exhaust-air ventilation system. The temperature of the animal facility was kept at 20-25° C. (±<3° C.) with relative humidity around 40-60%. The animal facility was lighting 12 hours every day with clean air flow of around grade 100, lighting for work area was around 150-300 LX, for caged animal area was 100-200 LX. Concentration of ammonia was less than 14 mg/m3, noise was less than 60 dB. Food and drinking water (deionized and ultrafiltered) were given freshly every day to mice.

### 3. Tumor Implantation

Human breast cancer-derived Bcap-37 strain cells were implanted into a BALB/c-nu mouse to make a Bcap-37 tumor-bearing host animal. In a biosafety cabinet under sterilization condition, Bcap-37 tumor was removed from the tumor-bearing host animal, and the tumor tissues were rinsed with 1×PBS. The tumor areas with fine growth without tumor decay were collected, and cut into small pieces of blocks in 2 mm³ with a scalpel. The tumor blocks were rinsed with 1×PBS, and implanted one by one underneath the skin of each mouse's right armpit with a tubing needle. Total 8 groups (ten animals per group) were prepared. Eleven days after implantation, tumors were grown to 110-120 mm³ in size.

### 4. Injection of Microvesicle (Cytomox)

The microvesicles were injected into the above-mentioned mice having the grown tumor. Intra-tumor injection of microvesicles was performed twice a week for three weeks, for each animal into tumors with injectable solutions containing isolated microvesicles. That is, total six injections were performed.

For both Cytomox HD and Cytomox EX, the first 3 injections were done at a lower dosage ranging from 10.0 to 30.0 μg protein/kg body weight, and the subsequent 3 injections were done at a higher dosage of 1.0 to 3.0 mg protein/kg body weight. The group into which Cytomox HD were injected at 10.0 μg protein/kg body weight and subsequently 1.0 mg protein/kg body weight is herein referred to as low dose Cytomox HD injection group. The group into which Cytomox HD were injected at 30.0 μg protein/kg body weight and subsequently 3.0 mg protein/kg body weight is herein referred to as high dose Cytomox HD injection group. Further, other two groups into which Cytomox EX was injected in a similar manner are herein referred to as low dose Cytomox EX injection group and high dose Cytomox EX injection group respectively. For Cytomox p53 and Cytomox PTEN, the first 3 injections were done at 10.0 μg protein/kg body weight and the subsequent 3 injections were done at a higher dosage of 1.0 mg protein/kg body weight (Cytomox p53 injection group and Cytomox PTEN injection group). As positive control, recombinant interferon α-2b was injected at 250×10⁴ IU/kg body weight, twice a week for three weeks (positive control group). As negative control, 1×PBS was injected to tumors of the tumor-bearing mice twice a week for three weeks (negative control group). Animals in each group were put for euthanasia by cervical dislocation 48 hours after final dosage. The tumors were removed from the dead mice and fixed and processed for pathological examination.

As a result, in this test, there were no acute toxic effects on the testing animals even during high-dose treatments, nor was any animal died due to the injection of genetically-engineered microvesicles (Cytomox).

### 5. Determination of Body Weight, Tumor Weight and Tumor Size

The body weights of mice were measured prior to test sample injection (tumor-including body weight) and before the euthanasia. Further, the body weights of the dead mice were measured after their tumor were removed from the bodies (net body weights). The weights of the removed tumors were measured.

Based on the measured values of the tumor weights, the efficiency of tumor growth inhibition was calculated as follows.

Efficiency of tumor growth inhibition (I)(%)=[1−T/C]×100
T: Average tumor weight (g) in each group injected with each test sample (Cytomox or positive control)
C: Average tumor weight (g) in negative control group

The calculated efficiencies of tumor growth inhibition were shown in Table 1.

TABLE 1

| | Dose*/ | | Efficiency of |
|---|---|---|---|
| | (injection/kg | Tumor weight | tumor growth |
| Test sample | body weight) | (g ± SD) | inhibition (%) |
| negative control | — | 1.73 ± 1.11 | |
| IFN α-2b | 250 × 10⁴ IU | 1.45 ± 0.58 | 16.06 |
| Cytomox HD | 3 mg | 1.41 ± 0.66 | 18.37 |
| Cytomox HD | 1 mg | 1.48 ± 0.61 | 14.67 |
| Cytomox EX | 3 mg | 1.36 ± 0.57 | 21.32 |
| Cytomox EX | 1 mg | 1.68 ± 0.40 | 2.83 |
| Cytomox p53 | 1 mg | 1.48 ± 0.67 | 14.73 |
| Cytomox PTEN | 1 mg | 1.57 ± 0.59 | 9.13 |

*Dose represents the dosage amount of 3 injections in second half.

As seen in Table 1, the direct injection into tumor foci of recombinant interferon α-2b caused inhibition of tumor growth by 16.06%. Intra-tumor injection with Cytomox HD showed inhibition of tumor growth by 18.37% at high dosage (3 mg protein/injection/kg body weight) and by 14.67% at low dosage (1 mg protein/injection/kg body weight). Thus, dose-dependent increase of tumor growth inhibitory effect was observed. Similarly, intra-tumor injection with Cytomox EX showed inhibition of tumor growth by 21.32% at high dosage (3 mg protein/injection/kg body weight) and by 2.83% at low dosage (1 mg protein/injection/kg body weight), and dose-dependent increase of the tumor growth inhibitory effect was also observed. Intra-tumor injection with Cytomox p53 or Cytomox PTEN also resulted in inhibition of tumor growth. These results indicate that the genetically-engineered microvesicles carrying transgene products can enter cells and functions of the transgene products can be transmitted to tumor cells.

In addition, the measurement of length and width of each tumor in mouse was done twice a week after the injection of test samples. A standard caliper was used to take the measurement. Based on the measured values, tumor volumes were calculated as follows.

Tumor volume (mm³)=(Tumor length×Tumor width)²/2

In high dose Cytomox HD injection group, high dose Cytomox EX injection group, and Cytomox PTEN injection group, after 4 days of injection of test samples (i.e., after 15 days of tumor implantation), the increase of tumor volume was continuously inhibited compared to that of negative control group to the last day of the measurement (i.e., at 31 day after tumor implantation and after 20 days of injection of test samples). Also in Cytomox p53 injection group, the increase of tumor volume was significantly inhibited after 12 days of injection of the test sample. In particular in high dose Cytomox HD injection group and high dose Cytomox EX injection group, the differences of tumor volumes between the groups and negative control group were increased over time. At 31 day after tumor implantation (the last day of the measurement), the tumor volume is 1,250 mm³ for negative control (PBS); about 1,000 mm³ for Interferon-α 2b; about 950 mm³ for Cytomox HD (evmp 130001, high dosage); about 1,000 mm³ for Cytomox p53 (emvp 130003, high dosage); about 1,100 mm³ for Cytomox PTEN (emvp 130006, high dosage); and about 1,000 mm3 for Cytomox EX (T130075, high dosage).

### 6. Pathomorphological Evaluation of Tumor

The removed tumors were subjected to pathological examination to evaluate their molphology. The evaluation criterion is as follows:

“−”, there is necrosis in the central area of the tumor foci, but there is no fibrosis;

“+”, there is less degree of inflammation in tumor tissues, with low grade of fibrosis;

“++”, there is inflammation in tumor tissues and decay of the tumor tissues, and fibrosis is observed all over the tumor; and

“+++” there is significant inflammation with tumor tissue decay and the highest grade of fibrosis is observed all over the tumor.

The results are shown in Table 2.

TABLE 2

| | | IFN α-2b | HD | HD | P53 | PTEN | EX | EX |
|---|---|---|---|---|---|---|---|---|
| Mouse | Negative | 250 × 10⁴ | 1 mg/k | 3 mg/k | 1 mg/k | 1 mg/k | 1 mg/k | 3 mg/k |
| no. | control | IU/kg | g | g | g | g | g | g |
| 1 | − | + | ++ | + | ++ | + | ++ | +++ |
| 2 | + | ++ | + | +++ | + | + | + | ++ |
| 3 | − | + | + | + | ++ | + | ++ | +++ |
| 4 | + | ++ | ++ | ++ | + | + | + | + |
| 5 | + | +++ | + | +++ | + | ++ | + | ++ |
| 6 | − | ++ | + | + | ++ | + | + | +++ |
| 7 | − | ++ | + | + | + | ++ | ++ | ++ |
| 8 | + | +++ | ++ | ++ | + | + | + | + |
| 9 | + | + | + | + | + | ++ | + | +++ |
| 10 | + | ++ | + | + | ++ | + | ++ | + |

As seen in Table 2, unlike the negative control, the administration of microvesicles carrying a tumor suppressor gene product into tumor foci frequently caused decay of tumor tissue and enhance inflammation and fibrosis in the tumor. In particular, the high dose injection of Cytomox HD or Cytomox ED to the tumor foci resulted in inflammation and decay of tumor tissues at higher levels and fibrosis over a wide range, which are different from tumor necrosis resulted from the lack of blood vessel angiogenesis in the central areas of tumor masses. It was considered that the decayed tumor tissues became fibrosis, which blocked the tumor expansion.

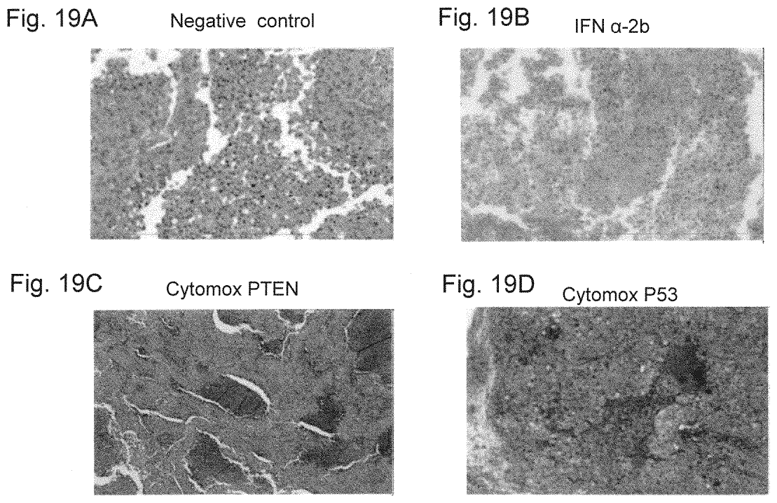

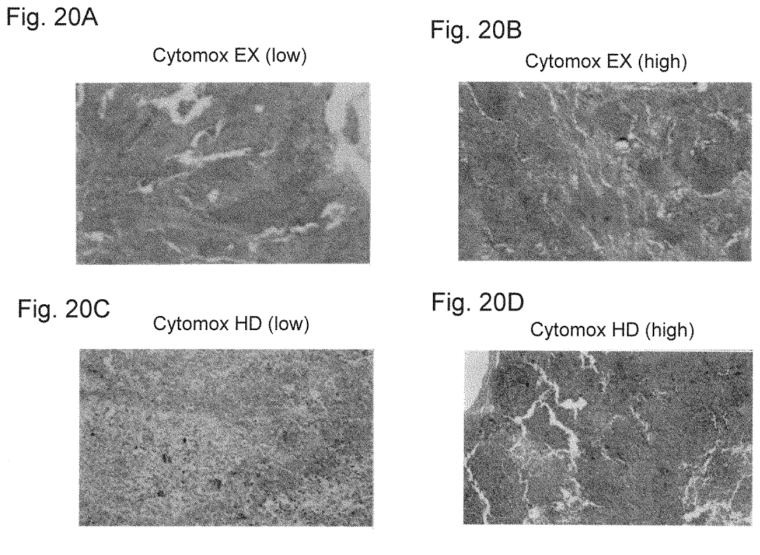

FIGS. 19A-D and 20A-D show typical morphologies observed for tumors from respective groups by pathological examination.

### 7. Western Blotting Analysis of Tumor

Protein extraction was performed from the removed tumors of Cytomox PTEN injection group and negative control group according to conventional methods, and PTEN protein therein was detected by Western blotting analysis as described in Example 13. As a result, PTEN was detected in both the tumor of the negative control group (microvesicle-uninjected tumor tissues) and the tumor of the microvesicle injection group (microvesicle-injected tumor tissues), and the levels of PTEN in the microvesicle-injected tumor tissues, however, were higher by two times than those of the microvesicle-uninjected tumor tissues. This indicates that the efficiencies of cell entry by the microvescles and of delivery of transgenes/transgene products via the microvescles are sufficiently high. In addition, as seen from the result, with the increased PTEN levels in the Cytomox PTEN injected tumor cells, the growth of tumor was inhibited (see, 9.13% inhibition in Table 1).

### INDUSTRIAL APPLICABILITY

The present invention is useful for the efficient production of genetically engineered microvesicles. The microvesicles according to the present application can be used for delivering biological substances to cells.

All publications, patents, and patent applications cited herein are incorporated herein by reference in their entirety.

1-21. (canceled)

22. A method for producing microvesicles comprising at least one selected from the group consisting of: (a) a product of a transgene and (b) a lentiviral RNA comprising the transgene, comprising:
(i) culturing a human cell into which the transgene has been introduced using a lentiviral vector in vitro to extracellularly release microvesicles from the human cell,
wherein said lentiviral vector is deficient in at least one structural protein gene and comprises the transgene under control of a human telomerase reverse transcriptase (TERT) gene promoter in a lentiviral genome sequence, and
wherein said lentiviral vector is a DNA vector encoding an RNA comprising the lentiviral genome sequence, or a viral particle carrying an RNA comprising the lentiviral genome sequence; and
(ii) collecting the released microvesicles.

23. The method according to claim 22, wherein said human cell into which the transgene under control of TERT gene promoter in a lentiviral vector has been introduced does not have said at least one structural protein gene.

24. The method according to claim 22, wherein said lentiviral vector is deficient in env gene.

25. The method according to claim 22, wherein said human TERT gene promoter comprises the nucleotide sequence of SEQ ID NO: 1.

26. The method according to claim 22, wherein said lentiviral genome sequence is an HIV genome sequence.

27. The method according to claim 22, wherein said transgene is a tumor-suppressor gene.

28. The method according to claim 27, wherein said tumor-suppressor gene is PTEN or p16 gene.

29. The method according to claim 22, wherein said transgene encodes a shRNA.

30. The method according to claim 29, wherein said shRNA targets a gene encoding a cell proliferation regulator.

31. The method according to claim 30, wherein said cell proliferation regulator is CDC6.

32. The method according to claim 22, wherein said human cell into which the transgene under control of TERT gene promoter in a lentiviral vector has been introduced is a kidney-derived cell.

33. A method for producing a pharmaceutical composition, comprising producing microvesicles by the method according to claim 27, and preparing a pharmaceutical composition comprising the microvesicles.

34. A method for producing a pharmaceutical composition, comprising producing microvesicles by the method according to claim 30, and preparing a pharmaceutical composition comprising the microvesicles.

35. A microvesicle comprising at least one selected from the group consisting of: (a) a product of transgene and (b) a lentiviral RNA comprising the transgene under control of TERT gene promoter in a lentiviral genome sequence.


SEQUENCE LISTING (27 sequences)

SEQ ID NO: 1 - DNA, 1389 bp, Homo sapiens
  The nucleotide sequence of the TERT gene promoter
gacaattcacaaacacagccctttaaaaaggcttagggatcactaaggggatttctagaagagcgacctgtaatcctaagtatttacaagacgaggctaacctccagcgagcgtgacagcccagggagggtgcgaggcctgttcaaatgctagctccataaataaagcaatttcctccggcagtttctgaaagtaggaaaggttacatttaaggttgcgtttgttagcatttcagtgtttgccgacctcagctacagcatccctgcaaggcctcgggagacccagaagtttctcgcccccttagatccaaacttgagcaacccggagtctggattcctgggaagtcctcagctgtcctgcggttgtgccggggccccaggtctggaggggaccagtggccgtgtggcttctactgctgggctggaagtcgggcctcctagctctgcagtccgaggcttggagccaggtgcctggaccccgaggctgccctccaccctgtgcgggcgggatgtgaccagatgttggcctcatctgccagacagagtgccggggcccagggtcaaggccgttgtggctggtgtgaggcgcccggtgcgcggccagcaggagcgcctggctccatttcccaccctttctcgacgggaccgccccggtgggtgattaacagatttggggtggtttgctcatggtggggacccctcgccgcctgagaacctgcaaagagaaatgacgggcctgtgtcaaggagcccaagtcgcggggaagtgttgcagggaggcactccgggaggtcccgcgtgcccgtccagggagcaatgcgtcctcgggttcgtccccagccgcgtctacgcgcctccgtcctccccttcacgtccggcattcgtggtgcccggagcccgacgccccgcgtccggacctggaggcagccctgggtctccggatcaggccagcggccaaagggtcgccgcacgcacctgttcccagggcctccacatcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcga

SEQ ID NO: 2 - DNA, 429 bp, Homo sapiens
  The nucleotide sequence of 5′ part of TERT transcribed region
gtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgggcctccccggggtcggcgtccggctggggttgagggcggccggggggaaccagcgacatgcggagagcagcgcaggcgactcagggcgcttcccccgcaggtgtcctgcctgaaggagctggtggcccgagtgctgcagaggct

SEQ ID NO: 3 - DNA, 1818 bp, Homo sapiens
  The nucleotide sequence comprising the TERT gene promoter and 5′ part of TERT transcribed region
gacaattcacaaacacagccctttaaaaaggcttagggatcactaaggggatttctagaagagcgacctgtaatcctaagtatttacaagacgaggctaacctccagcgagcgtgacagcccagggagggtgcgaggcctgttcaaatgctagctccataaataaagcaatttcctccggcagtttctgaaagtaggaaaggttacatttaaggttgcgtttgttagcatttcagtgtttgccgacctcagctacagcatccctgcaaggcctcgggagacccagaagtttctcgcccccttagatccaaacttgagcaacccggagtctggattcctgggaagtcctcagctgtcctgcggttgtgccggggccccaggtctggaggggaccagtggccgtgtggcttctactgctgggctggaagtcgggcctcctagctctgcagtccgaggcttggagccaggtgcctggaccccgaggctgccctccaccctgtgcgggcgggatgtgaccagatgttggcctcatctgccagacagagtgccggggcccagggtcaaggccgttgtggctggtgtgaggcgcccggtgcgcggccagcaggagcgcctggctccatttcccaccctttctcgacgggaccgccccggtgggtgattaacagatttggggtggtttgctcatggtggggacccctcgccgcctgagaacctgcaaagagaaatgacgggcctgtgtcaaggagcccaagtcgcggggaagtgttgcagggaggcactccgggaggtcccgcgtgcccgtccagggagcaatgcgtcctcgggttcgtccccagccgcgtctacgcgcctccgtcctccccttcacgtccggcattcgtggtgcccggagcccgacgccccgcgtccggacctggaggcagccctgggtctccggatcaggccagcggccaaagggtcgccgcacgcacctgttcccagggcctccacatcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgggcctccccggggtcggcgtccggctggggttgagggcggccggggggaaccagcgacatgcggagagcagcgcaggcgactcagggcgcttcccccgcaggtgtcctgcctgaaggagctggtggcccgagtgctgcagaggct

SEQ ID NO: 4 - DNA, 14825 bp, Artificial sequence
  Recombinant plasmid pNL4-3 clone comprising the nucleotide sequence of full-length genomic DNA of HIV -1 NL4-3 strain
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcctagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacccaggaggtagaggttgcagtgagccaagatcgcgccactgcattccagcctgggcaagaaaacaagactgtctaaaataataataataagttaagggtattaaatatatttatacatggaggtcataaaaatatatatatttgggctgggcgcagtggctcacacctgcgcccggccctttgggaggccgaggcaggtggatcacctgagtttgggagttccagaccagcctgaccaacatggagaaaccccttctctgtgtatttttagtagattttattttatgtgtattttattcacaggtatttctggaaaactgaaactgtttttcctctactctgataccacaagaatcatcagcacagaggaagacttctgtgatcaaatgtggtgggagagggaggttttcaccagcacatgagcagtcagttctgccgcagactcggcgggtgtccttcggttcagttccaacaccgcctgcctggagagaggtcagaccacagggtgagggctcagtccccaagacataaacacccaagacataaacacccaacaggtccaccccgcctgctgcccaggcagagccgattcaccaagacgggaattaggatagagaaagagtaagtcacacagagccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcctcaacgtcgtaagctcaagcgatcctcccacctcagcctgcctggtagctgagactacaagcgacgccccagttaatttttgtatttttggtagaggcagcgttttgccgtgtggccctggctggtctcgaactcctgggctcaagtgatccagcctcagcctcccaaagtgctgggacaaccggggccagtcactgcacctggccctaaaccataatttctaatcttttggctaatttgttagtcctacaaaggcagtctagtccccaggcaaaaagggggtttgtttcgggaaagggctgttactgtctttgtttcaaactataaactaagttcctcctaaacttagttcggcctacacccaggaatgaacaaggagagcttggaggttagaagcacgatggaattggttaggtcagatctctttcactgtctgagttataattttgcaatggtggttcaaagactgcccgcttctgacaccagtcgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccaggggaggcagagattgcagtaagctgagatcgcagcactgcactccagcctgggcgacagagtaagactctgtctcaaaaataaaataaataaatcaatcagatattccaatcttttcctttatttatttatttattttctattttggaaacacagtccttccttattccagaattacacatatattctatttttctttatatgctccagttttttttagaccttcacctgaaatgtgtgtatacaaaatctaggccagtccagcagagcctaaaggtaaaaaataaaataataaaaaataaataaaatctagctcactccttcacatcaaaatggagatacagctgttagcattaaataccaaataacccatcttgtcctcaataattttaagcgcctctctccaccacatctaactcctgtcaaaggcatgtgccccttccgggcgctctgctgtgctgccaaccaactggcatgtggactctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcaggcctccacctgggtcttggaatgtgtcccccgaggggtgatgactacctcagttggatctccacaggtcacagtgacacaagataaccaagacacctcccaaggctaccacaatgggccgccctccacgtgcacatggccggaggaactgccatgtcggaggtgcaagcacacctgcgcatcagagtccttggtgtggagggagggaccagcgcagcttccagccatccacctgatgaacagaacctagggaaagccccagttctacttacaccaggaaaggc

SEQ ID NO: 5 - DNA, 8733 bp, Artificial sequence
  The nucleotide sequence from 5′ LTR to 3′ LTR in pTHTK
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcctagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttatcgaattcctgcagcccgggggatccgagctcggtaccaagcttaagtttaaacgctagcatggcttcgtacccccgccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgataacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 6 - DNA, 1212 bp, Homo sapiens
  PTEN CDS
atgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctga

SEQ ID NO: 7 - DNA, 1683 bp, Homo sapiens
  CDC6 CDS
atgcctcaaacccgatcccaggcacaggctacaatcagttttccaaaaaggaagctgtctcgggcattgaacaaagctaaaaactccagtgatgccaaactagaaccaacaaatgtccaaaccgtaacctgttctcctcgtgtaaaagccctgcctctcagccccaggaaacgtctgggcgatgacaacctatgcaacactccccatttacctccttgttctccaccaaagcaaggcaagaaagagaatggtccccctcactcacatacacttaagggacgaagattggtatttgacaatcagctgacaattaagtctcctagcaaaagagaactagccaaagttcaccaaaacaaaatactttcttcagttagaaaaagtcaagagatcacaacaaattctgagcagagatgtccactgaagaaagaatctgcatgtgtgagactattcaagcaagaaggcacttgctaccagcaagcaaagctggtcctgaacacagctgtcccagatcggctgcctgccagggaaagggagatggatgtcatcaggaatttcttgagggaacacatctgtgggaaaaaagctggaagcctttacctttctggtgctcctggaactggaaaaactgcctgcttaagccggattctgcaagacctcaagaaggaactgaaaggctttaaaactatcatgctgaattgcatgtccttgaggactgcccaggctgtattcccagctattgctcaggagatttgtcaggaagaggtatccaggccagctgggaaggacatgatgaggaaattggaaaaacatatgactgcagagaagggccccatgattgtgttggtattggacgagatggatcaactggacagcaaaggccaggatgtattgtacacgctatttgaatggccatggctaagcaattctcacttggtgctgattggtattgctaataccctggatctcacagatagaattctacctaggcttcaagctagagaaaaatgtaagccacagctgttgaacttcccaccttataccagaaatcagatagtcactattttgcaagatcgacttaatcaggtatctagagatcaggttctggacaatgctgcagttcaattctgtgcccgcaaagtctctgctgtttcaggagatgttcgcaaagcactggatgtttgcaggagagctattgaaattgtagagtcagatgtcaaaagccagactattctcaaaccactgtctgaatgtaaatcaccttctgagcctctgattcccaagagggttggtcttattcacatatcccaagtcatctcagaagttgatggtaacaggatgaccttgagccaagaaggagcacaagattccttccctcttcagcagaagatcttggtttgctctttgatgctcttgatcaggcagttgaaaatcaaagaggtcactctggggaagttatatgaagcctacagtaaagtctgtcgcaaacagcaggtggcggctgtggaccagtcagagtgtttgtcactttcagggctcttggaagccaggggcattttaggattaaagagaaacaaggaaacccgtttgacaaaggtgtttttcaagattgaagagaaagaaatagaacatgctctgaaagataaagctttaattggaaatatcttagctactggattgccttaa

SEQ ID NO: 8 - DNA, 64 bp, Artificial sequence
  Oligonucleotide Cdc6-5-A
gatccccaggcacttgctaccagcaattcaagagattgctggtagcaagtgccttttttggaaa

SEQ ID NO: 9 - DNA, 64 bp, Artificial sequence
  Oligonucleotide Cdc6-3-A
agcttttccaaaaaaggcacttgctaccagcaatctcttgaattgctggtagcaagtgcctggg

SEQ ID NO: 10 - DNA, 60 bp, Artificial sequence
  DNA sequence which encodes CDC6 shRNA
cccaggcacttgctaccagcaattcaagagattgctggtagcaagtgccttttttggaaa

SEQ ID NO: 11 - DNA, 21 bp, Artificial sequence
  Primer B-NLR8950
  misc_feature (1)..(1): Biotynylated
gtgcctggctagaagcacaag

SEQ ID NO: 12 - DNA, 32 bp, Artificial sequence
  Oligonucleotide HD-A
cacgcgtcgcatcatatctccaggtgtgacag

SEQ ID NO: 13 - DNA, 37 bp, Artificial sequence
  Oligonucleotide HD-S
  misc_feature (36)..(37): n is a, c, g, or t
cctctgtcacacctggagatatgatgcgacgcgtgnn

SEQ ID NO: 14 - DNA, 19 bp, Artificial sequence
  Forward primer BHU5-S2
gagtgctcaaagtagtggt

SEQ ID NO: 15 - DNA, 22 bp, Artificial sequence
  Reverse primer HDA/SBOT
ctgtcacacctggagatatgat

SEQ ID NO: 16 - DNA, 24 bp, Artificial sequence
  Primer 5′ LTRU5
tctggctaactagggaacccactg

SEQ ID NO: 17 - DNA, 24 bp, Artificial sequence
  Primer 3′ NL5850
gctatgtcgacacccaattctgaa

SEQ ID NO: 18 - DNA, 23 bp, Artificial sequence
  Primer 3′ NL8960
tgtgcttctagccaggcacaagc

SEQ ID NO: 19 - RNA, 19 nt, Artificial sequence
  The nucleotide sequence of antisense sequence of CDC6 shRNA
uugcugguagcaagugccu

SEQ ID NO: 20 - RNA, 9 nt, Artificial sequence
  The nucleotide sequence of linker of CDC6 shRNA
uucaagaga

SEQ ID NO: 21 - DNA, 312 bp, Simian virus 40
agctttgcaaagatggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgaggtatttgcttcttccttaaatcctggtgttgatgcaatgtactgcaaacaatgg

SEQ ID NO: 22 - protein, 403 aa, Homo sapiens [sequence omitted]

SEQ ID NO: 23 - DNA, 471 bp, Homo sapiens
  CDS (1)..(471)
atggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattga

SEQ ID NO: 24 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 25 - DNA, 831 bp, Artificial
  pCMV p16INK4a
agtacggccgccagtgtgctggaattcaccaccatggagccttcggctgactggctggccacggccgcggcccggggtcgggtagaggaggtgcgggcgctgctggaggcgggggcgctgcccaacgcaccgaatagttacggtcggaggccgatccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagatatccccgattgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaagcatgctggggatgcggtgggctctatggcttctgaggcggaaagaaccagctggggctctagggggtatccccacgcgccctg

SEQ ID NO: 26 - DNA, 1182 bp, Homo sapiens
  CDS (1)..(1182)
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtgctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 27 - protein, 393 aa, Homo sapiens [sequence omitted]